SEQUENCE LISTING (3 sequences)

SEQ ID NO: 1 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 2 - DNA, 27 bp, Artificial Sequence
  Synthetic oligonucleotide
tgacaggatcctgaggaaacccagacc

SEQ ID NO: 3 - DNA, 33 bp, Artificial Sequence
  Synthetic oligonucleotide
cgcatggaagaagtagactaaggatccatatat

(19) United States
(12) Patent Application Publication
(10) Pub. No.: US 2016/0311795 A1
(43) Pub. Date: Oct. 27, 2016

(54) PYRIMIDINE DIAMINE DERIVATIVES AS INHIBITORS OF CYTOSOLIC HSP90

(71) Applicants: CALASIA PHARMACEUTICALS, INC., San Diego, CA (US); SANFORD-BURNHAM MEDICAL RESEARCH INSTITUTE, La Jolla, CA (US)

(72) Inventors: Sridhar G. Prasad, San Diego, CA (US); Nicholas Cosford, La Jolla, CA (US)

(21) Appl. No.: 14/790,111

(22) Filed: Jul. 2, 2015

Related U.S. Application Data
(63) Continuation of application No. 14/542,102, filed on Nov. 14, 2014, now abandoned.
Application No. 14/542,102 is a continuation of application No. PCT/US2013/000133, filed on May 15, 2013.
(60) Provisional application No. 61/647,081, filed on May 15, 2012.

(51) Int. Cl. C07D 401/12; A61K 31/5377; C07D 405/14; A61K 31/506

(52) U.S. Cl. CPC C07D 401/12; A61K 31/506; A61K 31/5377; C07D 405/14

(57) ABSTRACT

The present application describes organic compounds that inhibit the activity of cytosolic heat shock protein Hsp90. Also described are methods useful for prophylaxis, treatment, or amelioration of symptoms of diseases or conditions that are responsive to inhibition of Hsp90 activity, such as neurological diseases, proliferative disorders, and infection.

### RELATED APPLICATIONS

This application is a continuation of U.S. application Ser. No. 14/542,102 filed Nov. 14, 2014, which is a continuation of International Application No. PCT/US2013/000133, filed May 15, 2013, which claims the benefit of provisional application Ser. No. 61/647,081 filed May 15, 2012 entitled, “Novel, selective and potent brain-penetrating small molecule inhibitors of cytosolic Hsp90”. The entire teachings of the above applications are hereby incorporated herein by reference in their entirety.

### TECHNICAL FIELD

The invention relates to compounds having Hsp90 inhibitory activity for use in disease states responsive to inhibition of the heat shock protein Hsp90, and methods for using the compounds for treating the disease states.

### BACKGROUND

Heat shock proteins (Hsps) are produced by a cell in response to cellular stresses such as heat shock, oxidative stress, toxins, radiation, infection, and inflammation (Macario and de Macario 2000, Int. J. Clin. Lab. Res., 30:49-66). Heat shock proteins act as molecular chaperones by binding and stabilizing client proteins at intermediate stages of folding and allow proteins to fold to their functional states. Certain Hsps may also play a major molecular chaperone role under normal, stress-free conditions by regulating the correct folding, degradation, localization and function of a growing list of important cellular proteins. Hsp90 is one of the well-studied heat shock proteins. Two major human isoforms of Hsp90 are known, a major inducible form Hsp90a, and a minor constitutively expressed form Hsp90β. In addition, two other closely related chaperones, Endoplasmic reticulum GP96/GRP94, and mitochondrial TRAP1 (TNF receptor-associated protein 1). Little is known about the differences in function between Hsp90α/β, GRP94 and TRAP1 other than the differences in their sub-cellular localization.

Under normal conditions Hsp90 is the most abundant cytosolic heat shock protein in the cell. Hsp90 performs its chaperone function by interacting with a range of client and regulatory proteins (Smith, 2001, Molecular chaperones in the cell, pp. 165-178). Detailed insights into the chaperone function of Hsp90 have become available from biochemical and X-ray crystallographic studies (Prodromou et al., 1997, Cell, 90:65-75; Stebbins et al., 1997, Cell, 89:239-250). Hsp90 is isolated in complex with other chaperones including Hsp70, Hsc70 interacting protein (Hip), Hsp70-Hsp90 organizing protein (Hop), p23, and p50cdc37. Hsp90 has a distinct ATP binding site at its N-terminal end. According to a simplified model of the mechanism of function of Hsp90, binding of ATP to the amino terminal pocket alters Hsp90 conformation and permits association with a multi-chaperone complex. The multi-chaperone complex is formed by the binding of a client protein to an Hsp70/Hsp40 complex. The complex then associates with Hsp90 through the chaperone Hop. Upon replacement of ADP by ATP, the conformation of Hsp90 is altered, Hop and Hsp70 are released and another group of co-chaperones is recruited. ATP hydrolysis results in the release of these co-chaperones and the client protein from the mature complex. ATP binding site inhibitors herbimycin A, geldanamycin (GA) and 17-allylamino-17-desmethoxygeldanamycin (17-AAG) block the binding of ATP and prevent conversion to the mature complex (Grenert et. al., 1997. J Biol. Chem., 272:23834-23850). Herbimycin A and geldanamycin (GA) were shown to reverse the malignant phenotype of fibroblasts transformed by the v-Src oncogene (Uehara et al., 1986, Mol. Cell. Biol., 6:2198-2206), and to possess potent anti-tumor activity in both in vitro (Schulte et al., 1998, Cell Stress and Chaperones, 3:1008-108) and in vivo animal models (Supko et al., 1995, Cancer Chemother. Pharmacol., 36:305-315). By binding to the ATP binding site GA, and (17-N-allylamino-17-demethoxygeldanamycin) 17-AAG, inhibit the intrinsic ATPase activity of Hsp90 (Prodromou et al., 1997, Cell, 90:65-75; Stebbins et al., 1997, Cell, 89:239-250; Panaretou et al., 1998, EMBO J., 17:4829-4836).

Inhibition of Hsp90 ATPase activity results in the loss of p23 from the chaperone-client protein complex and interruption of the chaperone cycle. The resulting Hsp90-client protein complex is targeted for degradation by the ubiquitin proteasome pathway (Neckers et al., 1999, Invest. New Drugs, 17:361-373; Whitesell & Lindquist, 2005, Nat. Rev. Cancer, 5:761-772). Among the proteins that are targeted for degradation upon treatment with Hsp inhibitors are proteins involved in cell proliferation, cell cycle regulation and apoptosis, processes which are fundamentally important, and commonly deregulated in cancer, (Hostein et al., 2001, Cancer Res., 61:4003-4009). Therefore, modulation of Hsp90 activity may have potential benefit as an anticancer therapy.

Hsp90 client proteins are implicated in cell proliferation and survival, and therefore are important as targets for anticancer therapy they include, cellular Src (c-Src), a receptor tyrosine kinase, required for mitogenesis initiated by multiple growth factor receptors; ErbB2 (Her2/neu) a receptor tyrosine kinase overexpressed in a variety of malignancies including breast, ovarian, prostate, and gastric cancers; polo-like kinases (Plks), important as regulators of cell cycle progression during M-phase; Akt (PKB), which is involved in pathways that regulate cell growth by stimulating cell proliferation and suppressing apoptosis; c-Raf, B-Raf, and Mek which are involved in the RAS-RAF-MEK-ERK-MAP kinase pathway that mediates cellular responses to growth signals; EGFR, which is implicated in cell growth, differentiation, proliferation, survival, apoptosis, and migration; FMS-like tyrosine kinase 3 (FLT3), a receptor tyrosine kinase involved in cell proliferation, differentiation and apoptosis; c-met, a receptor tyrosine kinase which binds hepatocyte growth factor and regulates both cell motility and cell growth; Cdk1, Cdk2, Cdk4, and Cdk6 which drive the cell cycle; Wee-1, which is necessary for activation of the G2-phase checkpoint in response to DNA damage; P53, a tumor suppressor protein that causes cell cycle arrest and induces apoptosis; progesterone receptor, estrogen Receptor and androgen receptor; Hypoxia inducible factor-1a (HIF-1a), a transcription factor that controls the expression of genes which play a role in angiogenesis; and ZAP-70, a member of the Syk-ZAP-70 protein tyrosine kinase family normally expressed in T cells and natural killer cells, and expressed aberrantly in approximately 50% of cases of chronic lymphocytic leukemia (CLL). (U.S. 2011/0046155 A1, Feb. 24, 2011).

Correct folding of many proteins in vivo requires the assistance of heat-shock proteins acting as molecular chaperones. Cells which are stressed, for example, tumor cells which are surrounded by a hostile host environment, are heavily dependent upon this assistance. Tumor cells are observed to upregulate Hsps for maintaining the integrity of their proteomes under conditions which compromise protein folding Inhibitors of molecular chaperones in general and Hsp90 in particular have the potential to inhibit multiple aberrant signaling pathways simultaneously and, therefore hold promise as a class of chemotherapeutics with the unique ability to inhibit multiple aberrant signaling pathways simultaneously.

Treatment with anticancer agents further increases the stress imposed on the target tumor cells, and Hsps are implicated in resisting the effects of cancer drugs and treatment regimens for mitigating the deleterious effects of such stress. Therefore, modulators or inhibitors of chaperones, particularly Hsp90 inhibitors have potential as agents for sensitizing malignant cells to anticancer drugs and treatment regimens; alleviating or reducing the incidence of resistance to anticancer drugs and treatments; reversing resistance to anticancer drugs and or treatments;

potentiating the activity of anticancer drugs and or treatments; and delaying or preventing the onset of resistance to anticancer drugs and or treatments. (U.S. 2011/0046155 A1, Feb. 24, 2011).

Inhibitors of Hsp90 have potential for providing treatments for neurological diseases. In most neurodegenerative diseases, aberrant proteins accumulate in cells leading to pathological symptoms. For example, in Alzheimer's disease (AD), aggregation of hyperphosphorylated tau protein is implicated as one of the factors in the development of the disease. Hsp90 and its cofactor, the ubiquitin ligase (Carboxy terminus of Hsp70-interacting protein) CHIP, regulate levels of the microtubule-associated protein tau, and Hsp90 inhibitors are being pursued to clear tau aggregation for treating AD. (Calcul L. et al. 2012, Future Med. Chem. 4 (13):1751-61). Tau hyperphosphorylation is the product of deregulated Ser/Thr kinases such as cdk5. CDK5 phosphorylates several other neuronal proteins also, and is thought to play a role in the pathogenesis of neurodegenerative diseases other than AD such as, amyotrophic lateral sclerosis (ALS) and Niemann's Pick type-C disease (NPD). The activity of Cdk5 is regulated through association with neuron-specific activators, p35 and p39. (Tsai et al., Nature, 1994; 371:419-423). Conversion of p35 to p25 leads to aberrant Cdk5 activity. The p35 protein is a client protein for Hsp90. Inhibition of Hsp90 reduces the levels of p35. (Luo W. et al. Proc. Natl. Acad. Sci., 2007, 104 (22): 9511-9516) Inhibition of Hsp90 in cellular and mouse models of tauopathies leads to a reduction of the pathogenic activity of these proteins and results in elimination of aggregated Tau. (Luo W. et al. 2007).

Hsp90 inhibitor gledanamycin prevents alpha-synuclein mediated toxicity in several animal models of Parkinson's disease (PD) through upregulation of Hsp70 chaperone activity. The higher Hsp 70 chaperone activity prevents the formation of alpha-synuclein aggregates (Auluck, P K and Bonini, N M, 2002, Nat. Med. 8: 1185-1186; Fowler, T R., et. al., 2005, J. Mol. Biol. 351:1081-1100), and siRNA-mediated depletion of TRAP1 sensitizes cells of oxidative-stress-induced cytochrome c release and cell death, indicating a role for TRAP1 (mitochondrial Hsp90) in the modulation of the mitochondrial apoptotic cascade Inhibition of Hsp90 may ameliorate the cytotoxicity induced by these PD related proteins.

Several clinical trials of Hsp90 inhibitor drugs are ongoing for treatment of cancer. However, a number of trials have been abandoned, largely for lack of efficacy at maximum tolerated doses. Different cellular mechanisms have been reported to exist which may render cells less susceptible to the effects of Hsp90 inhibitor treatment. (Peter W. Piper P W and Millson S H, Pharmaceuticals 2011, 4, 1400-1422). For example, a major effect of Hsp90 inhibition is a strong induction of the heat shock response, a stress response that increases cellular levels of pro-survival chaperones such as Hsp27 and Hsp70. This response is not beneficial in the context of cancer treatment, but may be advantageous in the context of other disease conditions. Further, the inhibitors do not always access the Hsp90 proteins of the mitochondrion, which are forms of Hsp90 that in cancer cells operate to suppress apoptosis. In the case of neurodegenerative diseases, the inhibitor should be also effective at passing the blood brain barrier.

First generation of Hsp90 inhibitors based on geldanamycin, a benzoquinone ansamycin have several drawbacks including low solubility, hepatotoxicity as well as being substrates for the p-glycoprotein (P-gp) export pump involved in multi-drug resistance. Second generation Hsp90 inhibitors also have significant liabilities or limitations including poor oralbioavailability, ocular toxicity, scaffolds that are not pharmaceutical-like.

For the reasons above, there is a need for developing Hsp90 inhibitors that are more effective pharmaceutical agents.

### SUMMARY

An embodiment of the invention provides a compound having formula (I)

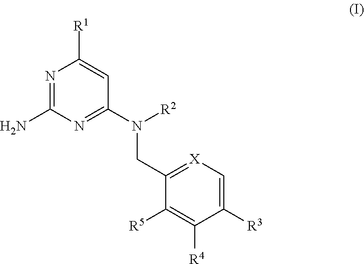

or a salt, hydrate, or solvate of the compound, such that
R¹ is H, CH₃, OCH₃, CF₃, F, Cl, Br, or I;
X is C, or N;
R² is H; CH₃; CH2CH₃; CH(CH₃)₂; C(CH₃)₃; CH₂CH₂CH₃; CH₂CH₂CH₂OH; CH₂CH₂CH₂CH₃; CH₂CH₂CH₂CH₃; CH₂CHCH₂; CH₂CH₂CH₂CH₂CH₂CH₃;

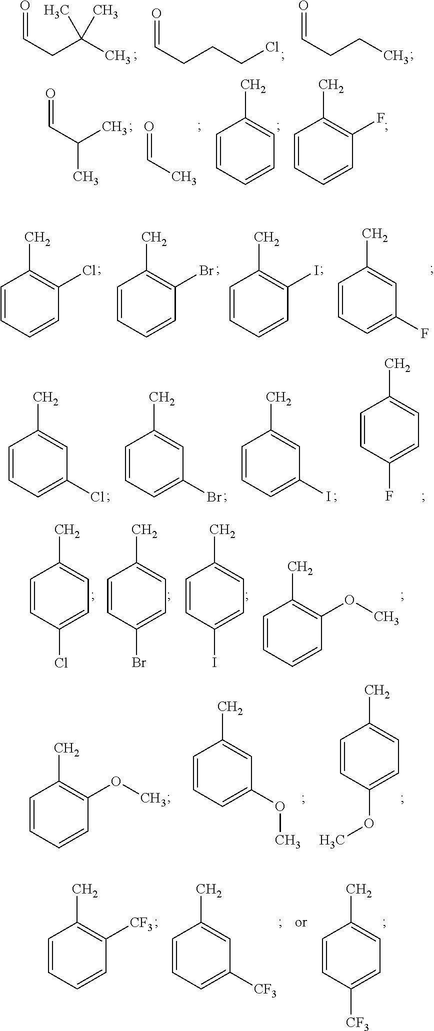

R³ is H; CH₃; OCH₃; F; Cl; Br; I; or CF_{3;}
R⁴ is H; CH₃; OCH₃; F; Cl; Br; I; or CF_{3;}
R⁵ is H; CH₃; OCH₃; F; Cl; Br; I; or CF_{3.}

In related embodiments the invention provides a compound having formula (I) such that R² is

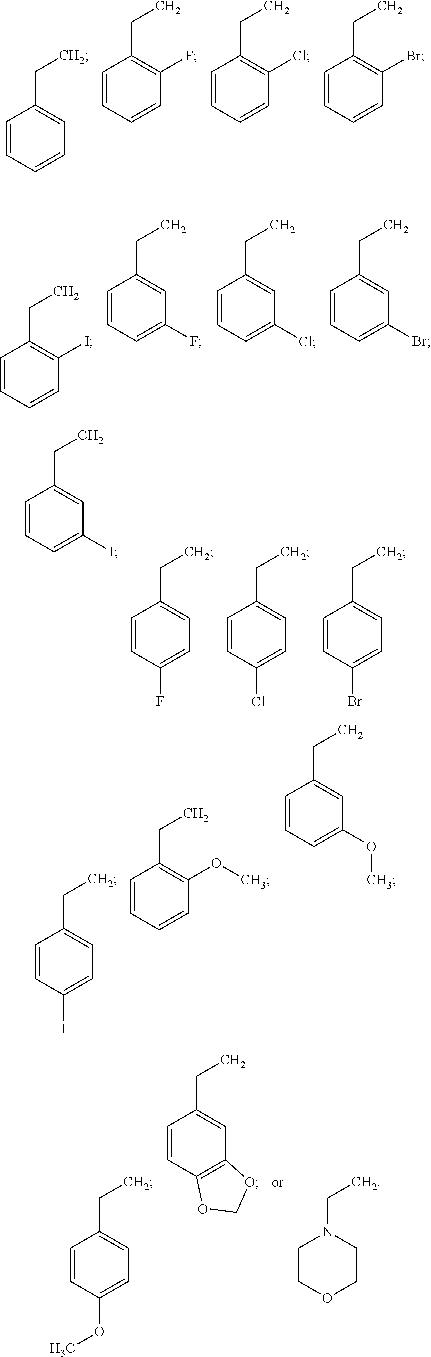

In a related embodiment the invention provides a pharmaceutical composition including at least one compound according to formula (I), together with one or more pharmaceutically acceptable carriers or excipients.

Another aspect of the invention provides a method for prophylaxis or treatment of a disease state or condition in a subject, such that the disease state or condition is responsive to inhibition of Hsp90 activity in the subject, the method including administering to the subject in need thereof, an amount of at least one compound according to formula (I) effective to inhibit the Hsp90 activity in the subject.

A related embodiment of the invention provides a method for prophylaxis or treatment of a disease state or condition in a subject, such that the disease state or condition is responsive to inhibition of Hsp90 activity in the subject, the method including administering to the subject in need thereof, an amount of at least one compound according to formula (I) effective to inhibit the Hsp90 activity, and an additional therapeutic agent.

In a related embodiment, the invention provides a method for alleviating or reducing the incidence of a disease state or condition in a subject, such that the disease state or condition is mediated by Hsp90 in the subject, the method including administering to the subject in need thereof, an amount of at least one compound according to formula (I) effective to inhibit the Hsp90 activity. In a related embodiment the invention provides a method for alleviating or reducing the incidence of a disease state or condition in a subject, such that the disease state or condition is mediated by Hsp90, the method including administering to the subject in need thereof, an amount of at least one compound according to formula (I) effective to inhibit the Hsp90 activity, and an additional therapeutic agent.

Another embodiment of the invention provides a method for prophylaxis or treatment of a disease state or condition in a subject undergoing treatment with a therapeutic agent, such that the disease state or condition is the development of resistance to the therapeutic agent, such that the disease state or condition is responsive to inhibition of Hsp90 in the subject, the method including: administering to the subject in need thereof, an amount of at least one compound according to formula (I) effective to inhibit the Hsp90 activity.

According to another embodiment the invention provides a method for alleviating or reducing the incidence of a disease state or condition in a subject undergoing treatment with a therapeutic agent, such that the disease state or condition is the development of resistance to the therapeutic agent, such that the disease state or condition is responsive to inhibition of Hsp90 in the subject, the method including: administering to the subject in need thereof, an amount of at least one compound according to formula (I) effective to inhibit the Hsp90 activity.

In related aspects of the methods of the invention, the Hsp90-mediated disease state or condition or disorder is selected from the group including an autoimmune disease, an inflammatory disease, a neurological disease, an infection, a cancer, a carcinoma, a cardiovascular disease, an allergy, asthma, a proliferative disorder, a metabolic disease, a leukemia, a neoplasm, a hormone-related disease, age-related macular degeneration, and tumors or symptoms resulting from neurofibromatosis. Neurofibromatosis includes neurofibromatosis type 1, which manifests itself in many forms including: small cutaneous neurofibromas; plexiform neurofibroma; freckling of the groin or the axilla; café au lait spots, which is pigmented, light brown macules located on nerves; skeletal abnormalities such as sphenoid dysplasia or thinning of the cortex of the long bones of the body; optic glioma or tumors on the optic nerve; scoliosis; and macrocephaly in pediatric population without hydrocephalus. Neurofibromatosis includes also neurofibromatosis type 2, which manifests itself in forms including: bilateral acoustic neuromas or schwannoma; headaches; facial weakness/paralysis; balance problems; and peripheral vertigo.

In other aspects of the methods of the invention, the Hsp90-mediated disease state or condition or disorder is a neurodegenerative disease selected from the group including Parkinson's disease, Alzheimer's disease, Huntington's disease, and Amyotrophic lateral sclerosis. In yet other aspects of the methods of the invention, the Hsp90-mediated disease state or condition or disorder is a fibrogenetic disorder selected from the group including liver cirrhosis, scleroderma, polymyositis, systemic lupus, rheumatoid arthritis, interstitial nephritis, pulmonary fibrosis, and keloid formation.

Another embodiment of the invention provides a method for treating a disease or condition including or arising from abnormal cell growth in a mammal, the method including administering to the mammal an amount of at least one compound according to formula (I) effective to inhibit Hsp90 activity in the mammal. A related embodiment provides a method for alleviating or reducing the incidence of a disease or condition including or arising from abnormal cell growth in a mammal, the method including administering to the mammal an amount of at least one compound according to formula (I) effective to inhibit Hsp90 activity in the mammal Another related embodiment provides a method for the prophylaxis or treatment of a disease state or condition having or arising from abnormal cell growth in a mammal, the method including administering to the mammal an amount of at least one compound according to formula (I) effective to inhibit Hsp90 activity in the mammal In related embodiments of the methods of the invention, the disease state or condition arising from abnormal cell growth includes a carcinoma of the bladder, breast, colon, kidney, epidermis, liver, lung, esophagus, gall bladder, ovary, pancreas, stomach, cervix, thyroid, prostate, gastrointestinal system, or skin; a hematopoietic tumor of lymphoid lineage; a hematopoietic tumor of myeloid lineage; thyroid follicular cancer; a tumor of mesenchymal origin; a tumor of the central or peripheral nervous system; melanoma; seminoma; teratocarcinoma; osteosarcoma; xeroderma pigmentosum; neurofibromatosis; keratoacanthoma; thyroid follicular cancer; and Kaposi's sarcoma.

Another embodiment of the invention provides a method for alleviating or reducing the incidence of resistance to an anticancer drug in a subject including administering to a subject in need thereof, an amount of at least one compound according to formula (I) effective to inhibit Hsp90 activity in the subject. In a related embodiment the invention provides a method for reversing resistance to an anticancer drug including administering to a subject in need thereof, an amount of at least one compound according to formula (I) effective to inhibit Hsp90 activity in the subject. In another related embodiment the invention provides a method for potentiating the activity of an anticancer drug including administering to a subject in need thereof, an amount of at least one compound according to formula (I) effective to inhibit Hsp90 activity in the subject. According to yet another related embodiment, the invention provides a method for delaying or preventing the onset of resistance to an anticancer drug including administering to a subject in need thereof, an amount of at least one compound according to formula (I) effective to inhibit the Hsp90 activity in the subject.

### BRIEF DESCRIPTION OF THE DRAWINGS

FIG. 1 is a line and a bar graph showing amounts of a control Hsp90 inhibitor SNX-0723, and inhibition of Hsp90 activity by the compound, respectively, in the brain tissue, of a rat treated with the compound.

FIG. 1 panel A is a line graph showing concentration in micromoles (μM) of control Hsp90 inhibitor SNX-0723 (structure shown as inset) in central nervous system (CNS) and in plasma as a function of time in hours after dosing a rat at a dosing regimen of 10 milligrams/kilogram (mg/kg) weight of animal. This example demonstrates that Hsp90 inhibitor SNX-0723 selectively partitions into the CNS compartment compared to plasma.

FIG. 1 panel B is a bar graph showing percent inhibition of Hsp90 activity by the control Hsp90 inhibitor SNX-0723 in the brain tissue of a rat dosed at 10 mg/kg. The first bar and second bars show inhibitory effect at 2 and 8 hours post dosage. The black line on each bar represents the standard deviation in the measurement and the asterisks are the statistical significance of the measurement.

FIG. 2 is a bar graph and a table showing Hsp90 inhibitory activity of small molecules (fragments), which are to be used to synthesize a larger compound for inhibiting Hsp90 activity based on the inhibitory potency of the individual fragments.

FIG. 2 panel A is a bar chart in which each bar shows the number of fragments that have Hsp90 inhibitory activity within the range shown on the X-axis. From the left, the bars show that: seven fragments have 70-100% inhibitory effect; 35 fragments have 40-69% inhibitory effect; seven fragments have 20-39% inhibitory effect; and 142 fragments have 0-19% inhibitory effect.

FIG. 2 panel B is a table which shows molecular structure, molecular weight in Daltons, and physico-chemical properties of seven fragments (from panel A) that have inhibitory effect of 70-100% on the activity of Hsp90.

FIG. 3 shows the sequence of development of compounds possessing improved Hsp90 inhibitory activity based on three dimensional structures of the complexes of the compounds with Hsp90, and the pharmacological properties of the compounds. The stepwise improvement in the inhibitory effects of the compounds on the Hsp90 activity as measured by inhibitory constant Kᵢ of each compound is shown. The compounds have Kᵢ in the nanomolar (nM) range. The lower the value of Kᵢ the more potent is the compound.

FIG. 4 is a set of two graphs showing amounts (CNS exposure) of an exemplary di-substituted amine compound, and an exemplary tri-substituted amine compound in the CNS of rats treated with each compound.

FIG. 4 panel A shows an example of the concentration in the CNS of one of the di-substituted amine compounds as a function of time (hours) post dosing. The solid circles are concentrations in the CNS determined experimentally, and open circles represent estimates of concentrations in the CNS based on extrapolation of the concentrations of the compound in plasma.

FIG. 4 panel B shows an example of the concentration in the CNS of one of the tri-substituted amine compounds as a function of time (hours) post dosing. The solid circles are concentrations in the CNS determined experimentally, and open circles represent estimates of concentrations in the CNS based on extrapolation of the concentrations of the compound in plasma. The tri-substituted amine compound shows much greater CNS exposure compared to the di-substituted amine compound.

FIG. 5 is a set of bar graphs and a plot showing partitioning of an exemplary Hsp90 inhibitor SB-0639353 into the CNS, inhibition of Hsp90 by the compound, and effect of the compound on an exemplary biomarker of Hsp90 inhibition in rats treated with the compound.

FIG. 5 panel A is a bar graph showing preferential partitioning of an exemplary Hsp90 inhibitor SB-0639353 (also named SBI-0639353) into the CNS compared to the plasma. The levels of SB-0639353 in the CNS were determined by ex-vivo measurements using CNS tissues of rats.

FIG. 5 panel B is a bar graph showing inhibition of Hsp90 activity by the exemplary compound SBI-0639353 in the CNS tissues of rats when dosed at 40 mg/kg. The vertical line on the bar represents the standard deviation in the measurement.

FIG. 5 panel C is a bar graph showing the effect of Hsp90 inhibition by the exemplary compound SBI-0639353 on an in vivo biomarker for Hsp90, Akt1. SBI-0639353 was administered to the rats intraperitoneally. Akt1 kinase is a client protein that relies on Hsp90 activity for proper folding and maintenance in the cell Inhibition of Hsp90 activity by SBI-0639353 in rats in the CNS tissues results in degradation Akt1 and a decrease in its levels compared to vehicle treated rats. The vertical line on each bars represent the standard deviation in the measurement, and asterisks show the statistical significance of the measurement.

FIG. 6 is a concentration response curve of binding of FITC-labeled geldanamycin (GA-FITC) to Hsp90. A fluorescence polarization Hsp90 competitive binding assay was used to obtain the binding affinity of GA-FITC. Different concentrations of GA-FITC were used with or without 50 nM Hsp90. The K_{d} for GA-FITC was determined to be 3.1 nM with a Bmax of 188 nM.

FIG. 7 is a set two graphs showing inhibition of Hsp90 activity as a function of concentration of Hsp90 inhibitors.

FIG. 7 panel A is a graph of inhibition of Hsp90 activity as a function of concentration of SBI-0640725. The IC₅₀ for SBI-0640725 was determined to be 0.101 μM.

FIG. 7 panel B is a graph of inhibition of Hsp90 activity as a function of concentration of SBI-0639353. The IC₅₀ for SBI-0639353 was determined to be 0.255 μM.

FIG. 8 is a set of graphs showing programmed tumor cell death, variation in the levels of a cellular biomarker for Hsp90 inhibition as measured by levels of a client protein Akt1, and induction of Caspase-3, in tissue culture assays for measuring the activity of the exemplary Hsp90 inhibitory compound herein SBI-0639353.

FIG. 8 panel A is a graph of decrease in cell viability as a function of concentration of the exemplary compound SBI-0639353. The EC₅₀ for SBI-0639353 was determined to be 3.8 μM.

FIG. 8 panel B is a graph of Akt1 degradation as a result of inhibition of Hsp90 activity by increasing concentrations of SBI-0639353. The IC₅₀ for SBI-0639353 was determined to be 5.4 μM.

FIG. 8 panel C is a graph of induction of Caspase-3 by SBI-0639353 at 10 μM concentration. Caspase-3 induction was measured using the Caspase-3/7 assay kit (Promega, Inc. Madison, Wis. USA).

FIG. 9 is a graph showing the effects of inhibiting Hsp90 on Hsp70 in the CNS using the control compound SNX-0723 at a dosing regimen of 10 mg/kg. The level of Hsp70 increases in SNX-0723 compared to untreated rats (vehicle alone) and remains elevated up to 24 hrs (X-axis) post dosing.

FIG. 10 is a set of graphs showing reduction in tumor cell viability in response to treatment with SBI-0640725. FIG. 10 panels A, B and C show a reduction in cell viability of U251MG (human glioblastoma astrocytoma), MBA-MD-231 (breast cancer), and HepG2 (liver) cells respectively. Error bars indicate the SD of triplicate determinations.

FIG. 11 is a set of graphs showing an increase in cellular caspase levels in response to treatment with SBI-0640725. FIG. 11 panels A, B and C show an increase in caspase levels in U251MG, MBA-MD-231 and HepG2 cells respectively. Error bars indicate the SD of triplicate determinations.

FIG. 12 is a set of graphs showing degradation of Akt1, a known Hsp90 client protein, in response to treatment with SBI-0640725. FIG. 12 panels A and B show degradation of Akt1 in U251MG and MBA-MD-231 cells, respectively. Error bars indicate the SD of triplicate determinations.

FIG. 13 is a graph showing degradation of EGFR, a known Hsp90 client protein, in response to treatment with SBI-0640725 in U251MG cells. Error bars indicate the SD of triplicate determinations.

FIG. 14 is a graph showing induction of Hsp 70 in response to treatment with SBI-0640725 in U251MG (panel A) and MBA-MD-231 (panel B) cells. Error bars indicate the SD of triplicate determinations.

FIG. 15 is a set of two graphs showing the effect of SBI-0640725 on mitochondrial stress response in U251MG cells.

FIG. 15 panel A shows mitochondrial oxygen consumption rate (OCR) measured in real time before and after treating cells with increasing concentrations of SBI-0640725. The vertical line labeled D indicates the time of compound addition. After compound addition, cells were treated sequentially with 0.125 mg/ml oligomycin (O), 0.5 mM carbonyl cyanide 4-(trifluoromethoxy) phenylhydrazone (F) and 1 mM rotenone (R) while continuing measurement of the OCR.

FIG. 15 panel B shows the OCR data from FIG. 15 panel A, normalized by cell protein concentration, and after subtracting extra-mitochondrial respiration as determined by the respiration observed upon addition of rotenone. In FIG. 15 panel B, OA and FCCP are abbreviations for oligomycin and carbonyl cyanide-4-(trifluoromethoxy) phenylhydrazone, respectively. * p<0.05 (one-way ANOVA). Error bars indicate the SD of triplicate determinations.

FIG. 16 panel A is a fluorescence confocal microscopy image showing the effect of SBI-0640725 on mitochondrial integrity in U251MG cells using tetramethylrhodamine ethyl ester (TMRE) as the fluorescence indicator. FIG. 16 panel B is a graph showing quantitative changes in the TMRE fluorescence signal in cells treated with SBI-0640725 using a fluorimeter.

FIG. 17 is a graph showing the effect of SBI-0640725 on mitochondrial membrane potential in isolated mitochondria as a measure of mitochondrial integrity. Tetramethylrhodamine ethyl ester (TMRE)-loaded mitochondria isolated from U251MG cells were treated with 10 μM SBI-0640725 (left bar of each pair) or 2 μM (right bar of each pair) while continually monitoring changes in fluorescence intensity at 30° C. * p<0.05; ** p<0.001 (2-sided unpaired t-test). Error bars indicate the SD of triplicate determinations.

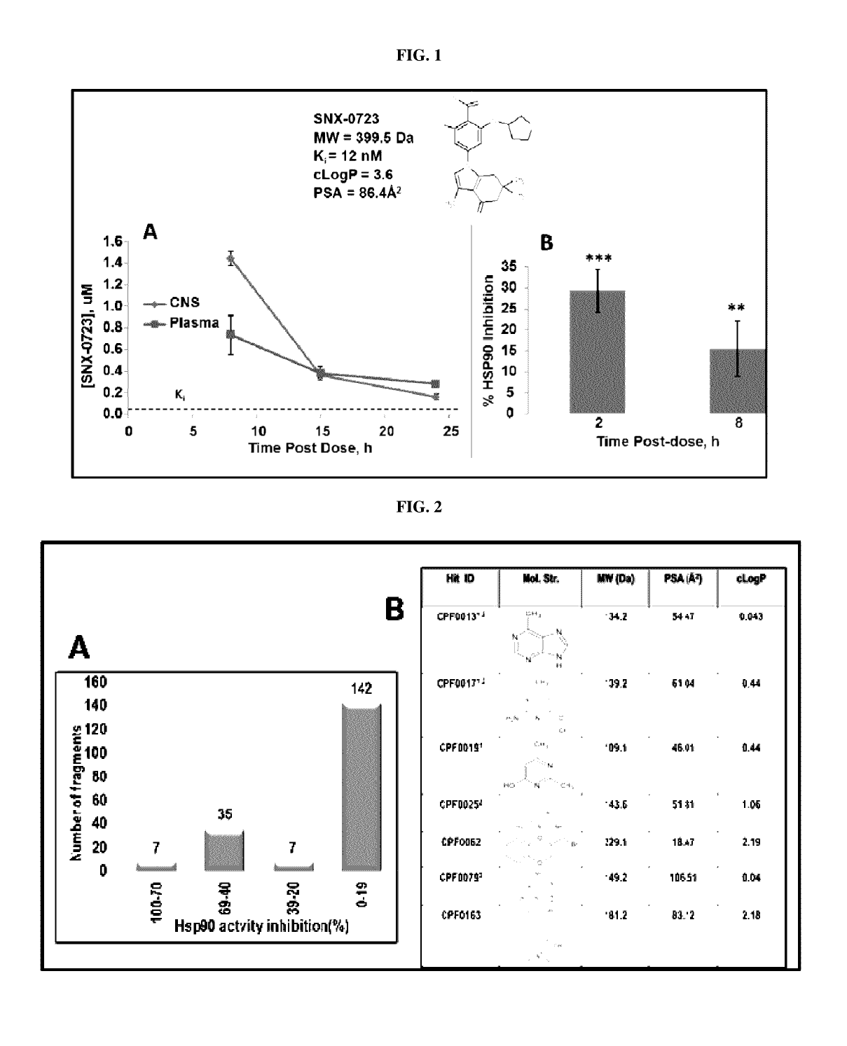

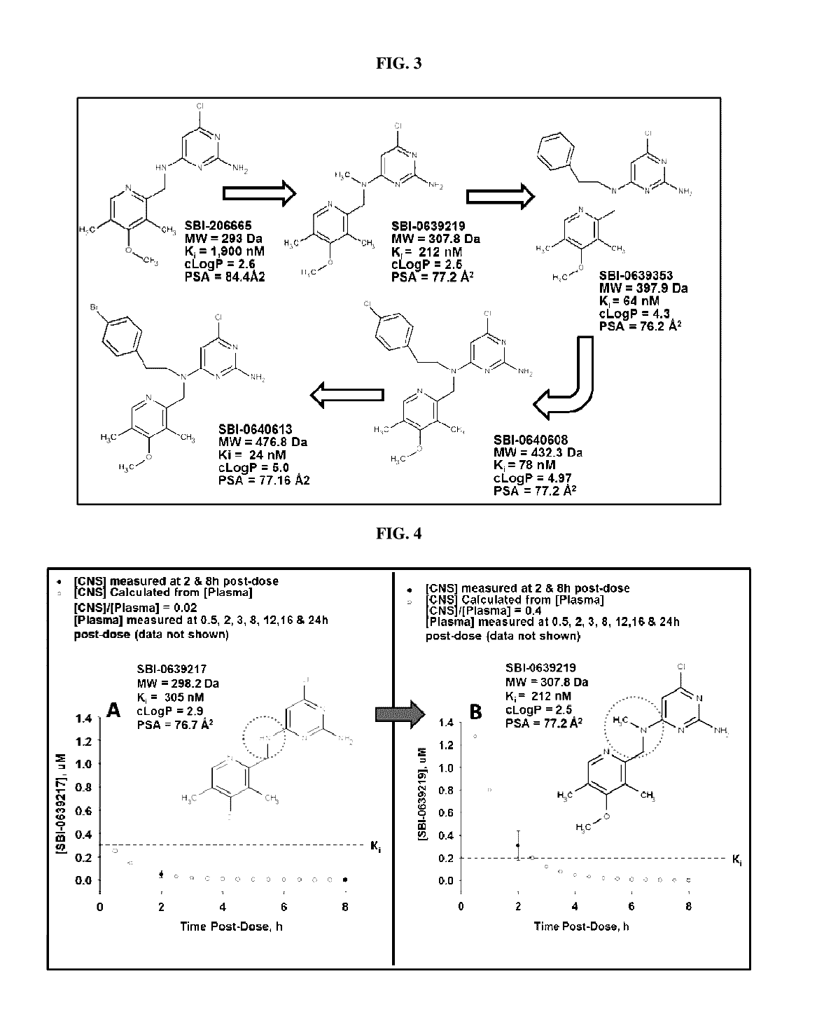

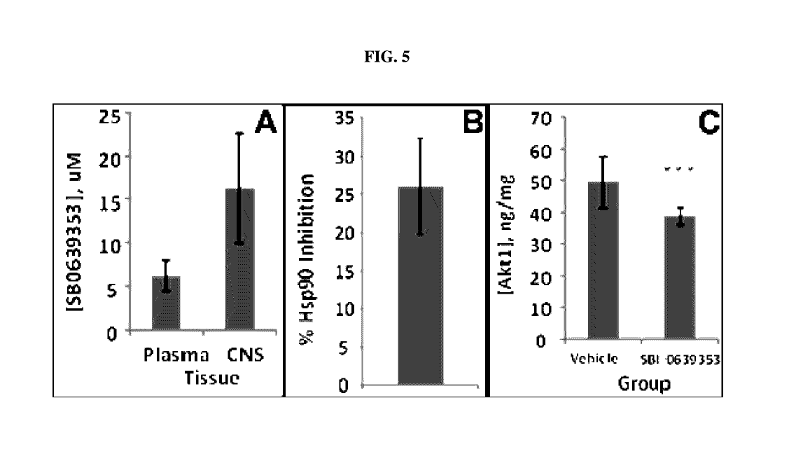

### DETAILED DESCRIPTION

Hsp90 is a molecular chaperone that assists client proteins to fold properly, stabilizes proteins against heat stress, and aids in protein degradation. Greater than 200 client proteins of Hsp90 have been identified. Hsp90 stabilizes a number of proteins required for tumor growth, such as proteins that are known to be involved in cell cycle regulation, signaling and chromatin-remodeling pathways. For this reason Hsp90 inhibitors are investigated as anti-cancer drugs. (Lu X et al. Biochemical Pharmacol. 2012, 83:8, 995-1004. Further, Hsp90 inhibitors act additively or synergistically with many other drugs in the treatment of both solid tumors and leukemias in murine tumor models and humans. (Lu X 2012). Hsp90 inhibitors potentiate the actions of anti-cancer drugs that target Hsp90 client proteins, including trastuzumab (Herceptin™) which targets Her2/Erb2B, as Hsp90 inhibition elicits the drug effects in cancer cell lines that are otherwise resistant to the drug (Modi S, et al. Clin Cancer Res. 2011; 17:5132-5139).

Hsp90 inhibitors described herein are effective in inhibiting the growth of cells derived from human prostate as shown in Example 32. It is envisioned that compounds herein are effective against a wide variety of tumor cells.

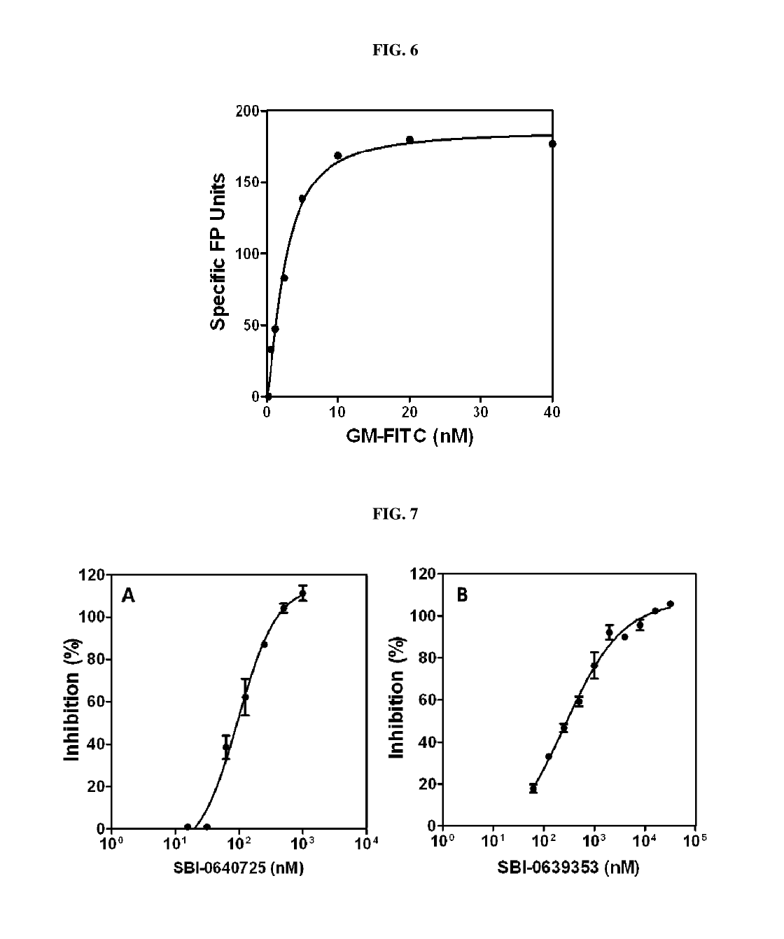

Various neurodegenerative disorders, including PD, Alzheimer's disease (AD), amyotrophic lateral sclerosis (ALS), Huntington disease (HD) and other polyglutamine expansion disorders, are associated with degeneration and death of specific neuronal populations due to accumulation of certain abnormal polypeptides or proteins (Meriin A B and Sherman M Y. Int J Hyperthermia. 2005; 21:5, 403-19). At least two components of cellular proteins are associated with PD: the ubiquitin proteasomal system (UPS) and the Hsps (Berke S J and Paulson H L. Curr Opin Genet Dev. 2003, 13:3, 253-61; Grunblatt E, et al. J Neural Transm. 2004, 111:12, 1543-73). Among the heat shock proteins Hsp90 is the main component of the cytosolic molecular chaperone complex, and has been implicated in the negative regulation of the heat shock factor 1 (HSF1), which is responsible for the transcriptional activation of the heat shock genes including Hsp40, Hsp70, and Hsp90 (Bharadwaj S et al. Mol Cell Biol. 1999, 19:12, 8033-41). Hsp90 forms a multichaperone complex with Hsp70 and Hsp40 to regulate several regulatory proteins including steroid hormone receptors and transcription factors. Hsp90 has been shown to be predominantly increased in PD brains, and the increase correlated with the elevated level of insoluble alpha-synuclein, a protein associated with the pathology of PD (Uryu K et al., Am J Pathol. 2006, 168:3, 947-61). Therefore, inhibition of Hsp90 is considered to be a promising approach for treatment of PD. A challenge to developing Hsp90 inhibitors for neurodegenerative disease is development of molecules that can efficiently cross the blood brain barrier. A number of Hsp90 inhibitors described herein, for example SBI-0639353, shows preferential partitioning into the brain compared to plasma (FIG. 6), and are therefore useful as molecules for treating or ameliorating the symptoms of PD.

The Hsp90 inhibitor GA has been tested as an agent for treatment of age-related macular degeneration. GA was found to attenuate the hypoxia-induced vascular endothelial growth factor expression in retinal pigment epithelium cells in vitro. (Wu, W C et al. Exp Eye Res. 2007 November, 85:5, 721-31). Hypoxia is the most common factor contributing to the pathogenesis of choroidal neovascularization, which is the major cause for blindness and occurs in proliferative diabetic retinopathy and age-related macular degeneration (AMD). Retinal pigment epithelial (RPE) cells play a role in the regulation of subretinal neovascularization under hypoxia. Significantly higher amount of the proangiogenic growth factor VEGF (vascular endothelial growth factor) was released from hypoxic RPE cells than from normoxic controls. Similarly VEGF (165) isoform gene expression was higher in hypoxic RPE compared to normoxic cells (Wu 2007). Pretreatment with GA significantly suppressed the hypoxia-induced VEGF gene expression in, and peptide release from the hypoxic RPE cells, indicating that Hsp90 inhibitors could be considered as novel anti-angiogenesis agents for diseases with intraocular neovascularization. (Wu, 2007). It is envisioned herein that Hsp90 inhibitors having the core formula (I) described herein will be effective as agents for treatment, or amelioration of the symptoms of AMD.

Hsp90 inhibitors described herein include those derived using the method of fragments based screening, which is a method used for finding lead compounds as part of the drug discovery process. It is based on identifying small chemical fragments, which may bind only weakly to the biological target, and then growing them or combining them to produce a lead with a higher affinity. Exemplary fragments tested herein for combining into a molecule for inhibiting Hsp90 are shown in FIG. 3.

Pyrimidine analogues described herein were synthesized following the general procedure described below.

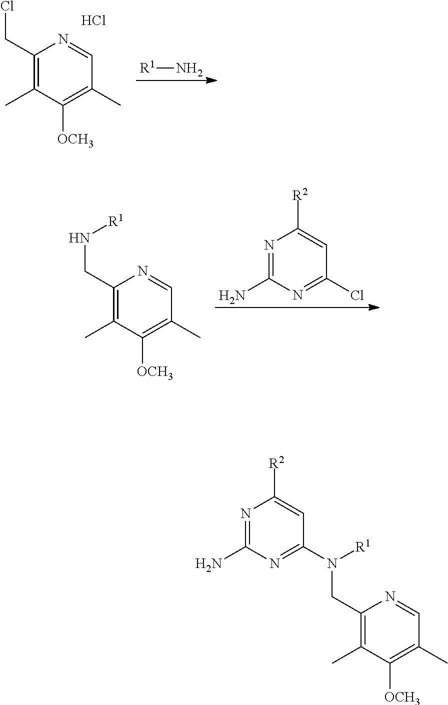

2-(Chloromethyl)-4-methoxy-3,5-dimethylpyridine hydrochloride, R¹—NH₂, and N,N-Diisopropylethylamine (DIPEA), were dissolved in DMF and heated by microwave irradiation at 125° C. for 10 min. The crude diamine was purified using automated preparative HPLC A suspension of the diamine and 4R²,6-chloro-pyrimidine-2-amine, and Et₃N was dissolved in DMF and heated at 65° C. for 2.5 h. The crude product was purified using automated preparative HPLC to yield the desired pyrimidine analogue.

The following general procedure was used for the synthesis of halogenated pyrimidine analogues described herein.

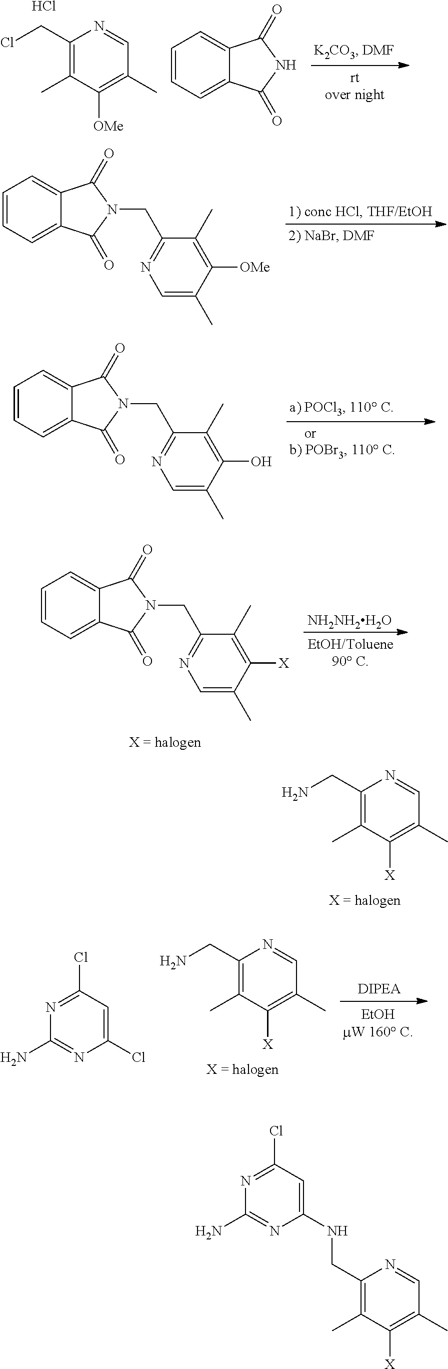

A suspension of 2-(chloromethyl)-4-methoxy-3,5-dimethylpyridine hydrochloride, pthalimide, and K₂CO₃ (25 g, 180.8 mmol) was made in DMF (200 mL) and reacted at room temperature for 16 h. To the resulting white solid, saturated NaHCO₃ was added until basic conditions were achieved, and the mixture filtered to obtain 2-((4-methoxy-3,5-dimethylpyridin-2-yl)methyl)isoindoline-1,3-dione as a white solid in quantitative yield. To a solution of the 2-((4-methoxy-3,5-dimethylpyridin-2-yl)methyl)isoindoline-1,3-dione in THF/EtOH concentrated HCl was added, and the reaction mixture was concentrated. The residue was dissolved in DMF, and NaBr was added, followed by heating at 120° C. for 1 h. EtOAc was added to the solution, and the precipitate obtained was filtered to obtain 2-((4-hydroxy-3,5-dimethylpyridin-2-yl)methyl)isoindoline-1,3-dione.

2-((4-Hydroxy-3,5-dimethylpyridin-2-yl)methyl)isoindoline-1,3-dione synthesized as shown above in and POCl₃/POBr₃ were combined in a sealed tube and heated at 110° C. for 45 min. The solution was cooled, added to ice water and basified with 40% KOH. The precipitate formed was filtered to obtain 2-((4-chloro/bromo-3,5-dimethylpyridin-2-yl)methyl)isoindoline-1,3-dione as a white solid. To a solution of 2-((4-chloro/bromo-3,5-dimethylpyridin-2-yl)methyl)isoindoline-1,3-dione in EtOH/Toluene NH₂NH₂.H₂O was added followed by heating at 90° C. for 20 min. The reaction mixture was cooled, filtered, and the filtrate was concentrated and washed with CH₂Cl₂ to obtain the crude amine. A suspension of 4,6-dichloropyrimidin-2-amine, DIPEA and (4-chloro/bromo-3,5-dimethylpyridin-2-yl)methanamine in EtOH was heated by microwave irradiation at 160° C. for 10 min. The crude product was purified using automated preparative HPLC to yield the desired product.

Table 1 below shows structure and formula weight of Hsp90 inhibitor compounds produced according to the methods above, and their inhibitory potencies.

TABLE 1
Molecular structure, formula weight, and Hsp90 inhibitory potency of pyrimidine
diamine derivatives

| | | | Biochemical Potency | | |
|---|---|---|---|---|---|
| | | Formula | | % | % |
| | | Weight | IC50 | Inhibition | Inhibition |
| Compound | Molecular Structure | (Da) | (μM) | at 10 μM | at 1 μM |
| SBI-0206664 | 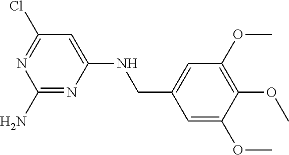 [SMILES: COc1cc(CNc2cc(Cl)nc(N)n2)cc(OC)c1OC] | 324.763 | | 25 | |
| SBI-0206665 | 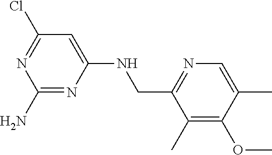 [SMILES: COc1c(C)cnc(CNc2cc(Cl)nc(N)n2)c1C] | 293.752 | 7.5 | 61 | 7 |
| SBI-0630160 | 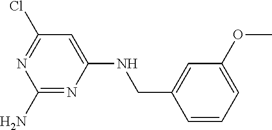 [SMILES: COc1cccc(CNc2cc(Cl)nc(N)n2)c1] | 264.711 | | 13 | |
| SBI-0630180 | 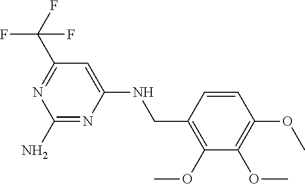 [SMILES: COc1ccc(CNc2cc(C(F)(F)F)nc(N)n2)c(OC)c1OC] | 358.32 | | 4 | |
| SBI-0633823 | 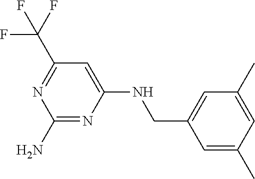 [SMILES: Cc1cc(C)cc(CNc2cc(C(F)(F)F)nc(N)n2)c1] | 296.291 | | 17 | |
| SBI-0633825 | 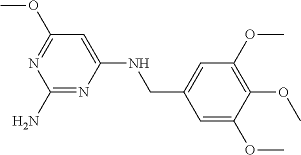 | 320.344 | >250 | | |
| SBI-0633826 | 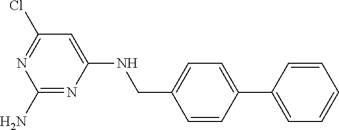 [SMILES: Nc1nc(Cl)cc(NCc2ccc(-c3ccccc3)cc2)n1] | 310.781 | | 21 | |
| SBI-0634911 | 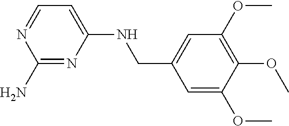 [SMILES: COc1cc(CNc2ccnc(N)n2)cc(OC)c1OC] | 290.318 | >250 | | |
| SBI-0634912 | 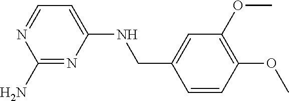 [SMILES: COc1ccc(CNc2ccnc(N)n2)cc1OC] | 260.292 | >250 | | |
| SBI-0635446 | 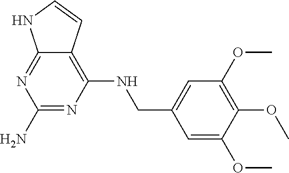 [SMILES: COc1cc(CNc2nc(N)nc3[nH]ccc23)cc(OC)c1OC] | 329.354 | | 10 | |
| SBI-0636438 | 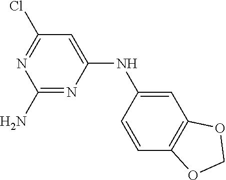 [SMILES: Nc1nc(Cl)cc(Nc2ccc3c(c2)OCO3)n1] | 264.668 | | 18 | |
| SBI-0638966 | 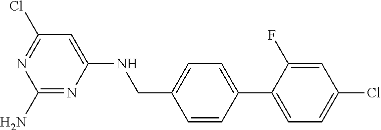 [SMILES: Nc1nc(Cl)cc(NCc2ccc(-c3ccc(Cl)cc3F)cc2)n1] | 363.216 | | 34 | |
| SBI-0638967 | 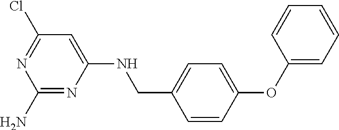 [SMILES: Nc1nc(Cl)cc(NCc2ccc(Oc3ccccc3)cc2)n1] | 326.78 | | 10 | |
| SBI-0638969 | 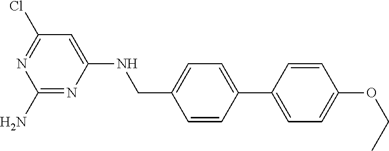 [SMILES: CCOc1ccc(-c2ccc(CNc3cc(Cl)nc(N)n3)cc2)cc1] | 354.833 | | 3 | |
| SBI-0639182 | 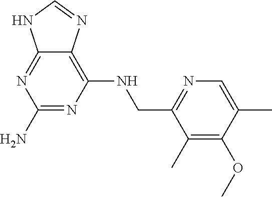 [SMILES: COc1c(C)cnc(CNc2nc(N)nc3[nH]cnc23)c1C] | 299.331 | | 19 | 4 |
| SBI-0639186 | 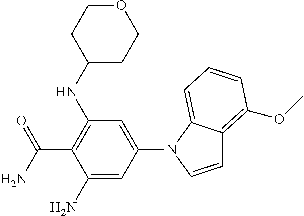 [SMILES: COc1cccc2c1ccn2-c1cc(N)c(C(N)=O)c(NC2CCOCC2)c1] | 383.416 | | 10 | 5 |
| SBI-0639217 | 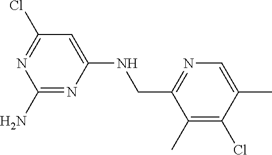 [SMILES: Cc1cnc(CNc2cc(Cl)nc(N)n2)c(C)c1Cl] | 298.171 | | 93 | 47 |
| SBI-0639218 | 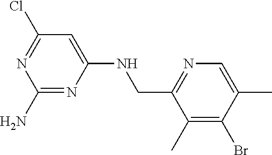 [SMILES: Cc1cnc(CNc2cc(Cl)nc(N)n2)c(C)c1Br] | 342.622 | | 94 | 44 |
| SBI-0639219 | 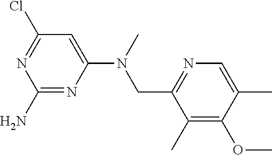 [SMILES: COc1c(C)cnc(CN(C)c2cc(Cl)nc(N)n2)c1C] | 307.779 | | 94 | 57 |
| SBI-0639220 | 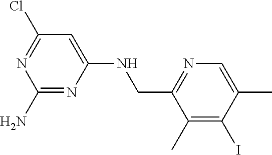 [SMILES: Cc1cnc(CNc2cc(Cl)nc(N)n2)c(C)c1I] | 389.622 | | 98 | 66 |
| SBI-0639349 | 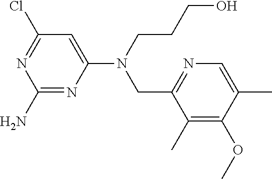 [SMILES: COc1c(C)cnc(CN(CCCO)c2cc(Cl)nc(N)n2)c1C] | 351.831 | 1.37 | | |
| SBI-0639350 | 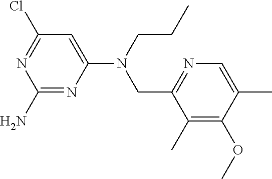 [SMILES: CCCN(Cc1ncc(C)c(OC)c1C)c1cc(Cl)nc(N)n1] | 335.832 | 1.1 | | |
| SBI-0639351 | 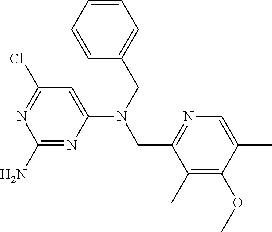 [SMILES: COc1c(C)cnc(CN(Cc2ccccc2)c2cc(Cl)nc(N)n2)c1C] | 383.875 | 2.21 | | |
| SBI-0639352 | 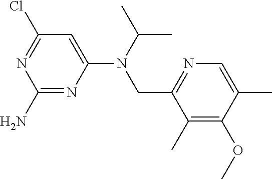 [SMILES: COc1c(C)cnc(CN(c2cc(Cl)nc(N)n2)C(C)C)c1C] | 335.832 | 32 | | |
| SBI-0639353 | 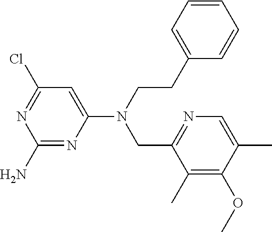 [SMILES: COc1c(C)cnc(CN(CCc2ccccc2)c2cc(Cl)nc(N)n2)c1C] | 397.901 | 0.255 | | |
| SBI-0639354 | 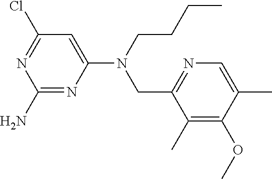 [SMILES: CCCCN(Cc1ncc(C)c(OC)c1C)c1cc(Cl)nc(N)n1] | 349.858 | 0.719 | | |
| SBI-0639355 | 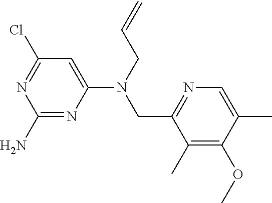 [SMILES: C=CCN(Cc1ncc(C)c(OC)c1C)c1cc(Cl)nc(N)n1] | 333.816 | 2.87 | | |
| SBI-0639899 | 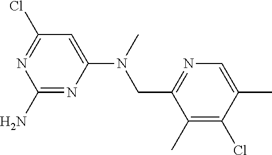 [SMILES: Cc1cnc(CN(C)c2cc(Cl)nc(N)n2)c(C)c1Cl] | 312.198 | 2.15 | | |
| SBI-0639900 | 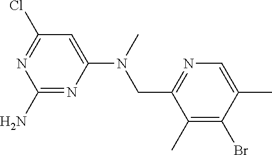 [SMILES: Cc1cnc(CN(C)c2cc(Cl)nc(N)n2)c(C)c1Br] | 356.649 | 32 | | |
| SBI-0639901 | 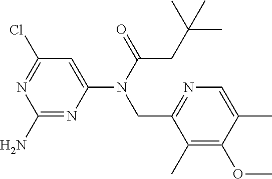 [SMILES: COc1c(C)cnc(CN(C(=O)CC(C)(C)C)c2cc(Cl)nc(N)n2)c1C] | 391.895 | 30 | | |
| SBI-0639902 | 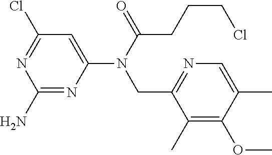 [SMILES: COc1c(C)cnc(CN(C(=O)CCCCl)c2cc(Cl)nc(N)n2)c1C] | 398.287 | 32 | | |
| SBI-0640492 | 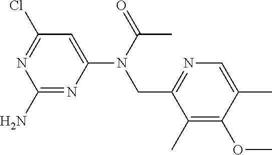 [SMILES: COc1c(C)cnc(CN(C(C)=O)c2cc(Cl)nc(N)n2)c1C] | 335.789 | 32 | | |
| SBI-0640605 | 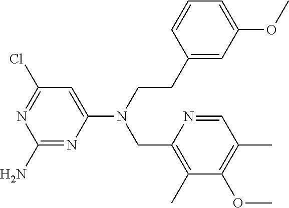 [SMILES: COc1cccc(CCN(Cc2ncc(C)c(OC)c2C)c2cc(Cl)nc(N)n2)c1] | 427.930 | 0.35 | | |
| SBI-0640606 | 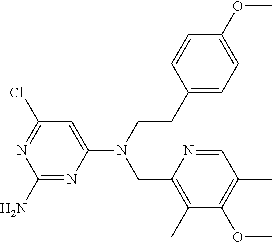 [SMILES: COc1ccc(CCN(Cc2ncc(C)c(OC)c2C)c2cc(Cl)nc(N)n2)cc1] | 427.930 | 0.432 | | |
| SBI-0640607 | 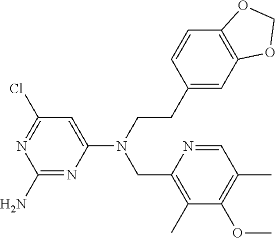 [SMILES: COc1c(C)cnc(CN(CCc2ccc3c(c2)OCO3)c2cc(Cl)nc(N)n2)c1C] | 441.910 | 0.368 | | |
| SBI-0640608 | 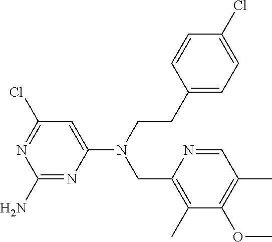 [SMILES: COc1c(C)cnc(CN(CCc2ccc(Cl)cc2)c2cc(Cl)nc(N)n2)c1C] | 432.350 | 0.311 | | |
| SBI-0640609 | 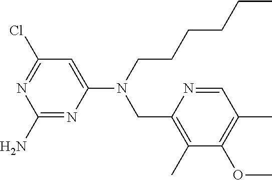 [SMILES: CCCCCCN(Cc1ncc(C)c(OC)c1C)c1cc(Cl)nc(N)n1] | 377.910 | 0.391 | | |
| SBI-0640610 | 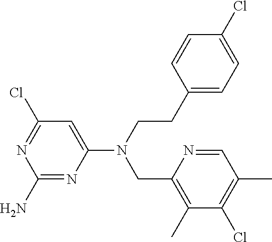 [SMILES: Cc1cnc(CN(CCc2ccc(Cl)cc2)c2cc(Cl)nc(N)n2)c(C)c1Cl] | 436.770 | 0.194 | | |
| SBI-0640611 | 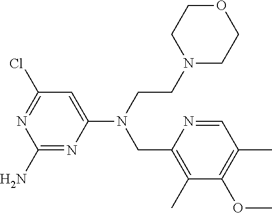 [SMILES: COc1c(C)cnc(CN(CCN2CCOCC2)c2cc(Cl)nc(N)n2)c1C] | 406.910 | 1.690 | | |
| SBI-0640612 | 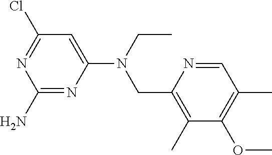 [SMILES: CCN(Cc1ncc(C)c(OC)c1C)c1cc(Cl)nc(N)n1] | 321.810 | 1.14 | | |
| SBI-0640613 | 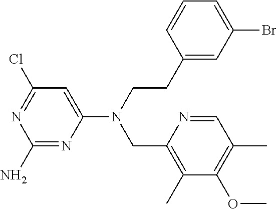 [SMILES: COc1c(C)cnc(CN(CCc2cccc(Br)c2)c2cc(Cl)nc(N)n2)c1C] | 476.80 | 0.097 | | |
| SBI-0640644 | 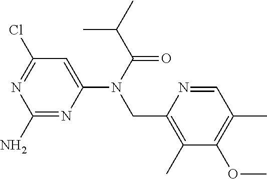 [SMILES: COc1c(C)cnc(CN(C(=O)C(C)C)c2cc(Cl)nc(N)n2)c1C] | 363.84 | 13.93 | | |
| SBI-0640645 | 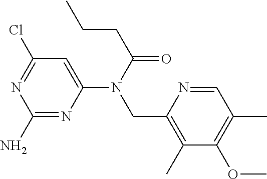 [SMILES: CCCC(=O)N(Cc1ncc(C)c(OC)c1C)c1cc(Cl)nc(N)n1] | 363.840 | 30.02 | | |
| SBI-0640725 | 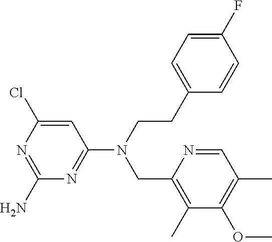 [SMILES: COc1c(C)cnc(CN(CCc2ccc(F)cc2)c2cc(Cl)nc(N)n2)c1C] | 415.890 | 0.101 | | |

Pharmaceutical Compositions

In one aspect of the present invention, pharmaceutical compositions are provided, wherein these compositions comprise at least one compound of formula (I), and optionally comprise a pharmaceutically acceptable carrier. In certain embodiments, these compositions optionally further comprise one or more additional therapeutic agents. In certain embodiments, the additional therapeutic agent or agents are selected from the group consisting of growth factors, anti-inflammatory agents, vasopressor agents, collagenase inhibitors, topical steroids, matrix metalloproteinase inhibitors, ascorbates, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Therapeutically Effective Dose

In yet another aspect, according to the methods of treatment of the present invention, treatment or alleviation of a disease state or condition in a subject responsive to inhibition of Hsp90 in the subject, including disease states or conditions preferentially responsive to inhibition of Hsp90 or a homolog thereof of an infectious agent in a subject suffering from infection, is promoted by contacting the subject with a therapeutically effective amount of a pharmaceutical composition as described herein. In certain embodiments of the present invention a “therapeutically effective amount” of the pharmaceutical composition is that amount effective for either treating, alleviating the symptoms of, reducing the incidence of or prophylaxix of a disease state or condition that is responsive to inhibition of Hsp90. The compositions, according to the method of the present invention, may be administered using any amount and any route of administration that is effective. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., extent of edema or hypervolemia; age, weight and gender of the patient; diet, time and frequency of administration, drug combinations, reaction sensitivities, and tolerance/response to therapy. The formulated pharmaceutical compositions might be administered every day, several times a day, every other day, every 3 to 4 days, every week, or once every two weeks depending on half-life and clearance rate of the particular formulation.

The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially in animal models, usually mice, rats, rabbits, dogs, pigs, or primates. The animal model is also used to achieve a desirable concentration range and route of administration. Such information can then be used to determine useful doses and routes for administration in humans. A therapeutically effective dose refers to an amount of active agent that ameliorates the symptoms or condition. Therapeutic efficacy and toxicity of active agents can be determined by standard pharmaceutical procedures in experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from animal studies are used in formulating a range of dosage for human use.

Administration of Pharmaceutical Compositions

After formulation with an appropriate pharmaceutically acceptable carrier in a desired dosage, the pharmaceutical compositions of this invention can be administered to humans or to other mammals as powders, ointments, or drops, by any route including without limitation orally, rectally, parenterally, intracistemally, intravaginally, intraperitoneally, bucally, ocularly, or nasally, depending on the severity and location of the edematous condition being treated. Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. Administration may be therapeutic or it may be prophylactic. The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions that are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions, which can be used include polymeric substances and waxes.

Uses of Pharmaceutical Compositions

The compositions herein comprising compounds having formula (I) are used to treat a large variety of disease or conditions including an autoimmune disease, an inflammatory disease, a neurological disease, an infection, a cancer, a carcinoma, a cardiovascular disease, an allergy, asthma, a proliferative disorder, a metabolic disease, a leukemia, a neoplasm, a hormone-related disease, age-related macular degeneration, and, tumors or symptoms resulting from neurofibromatosis. The compositions are also useful for treating fibrogenetic disorder selected from the group comprising liver cirrhosis, scleroderma, polymyositis, systemic lupus, rheumatoid arthritis, interstitial nephritis, pulmonary fibrosis, and keloid formation; and neurodegenerative disease selected from the group comprising Parkinson's disease, Alzheimer's disease, Huntington's disease, and Amyotrophic lateral sclerosis.

A skilled person will recognize many suitable variations of the compositions and methods to be substituted for or used in addition to those described above and in the claims. It should be understood that the implementation of other variations and modifications of the embodiments of the invention and its various aspects will be apparent to one skilled in the art, and that the invention is not limited by the specific embodiments described herein and in the claims. Therefore, it is contemplated to cover the present embodiments of the invention and any and all modifications, variations, or equivalents that fall within the true spirit and scope of the basic underlying principles disclosed and claimed herein.

The invention having now been fully described, it is exemplified by the following examples and claims which are for illustrative purposes only and are not meant to be further limiting.

### EXAMPLES

### Example 1

### 6-Chloro-N4-((4-methoxy-3,5-dimethylpyridin-2-yl)methyl)-N4-(4-methoxyphenethyl)pyrimidine-2,4-diamine

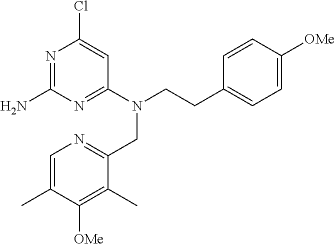 [SMILES: COc1ccc(CCN(Cc2ncc(C)c(OC)c2C)c2cc(Cl)nc(N)n2)cc1]

2-(Chloromethyl)-4-methoxy-3,5-dimethylpyridine hydrochloride (200 mg, 0.9 mmol), 2-(4-methoxyphenyl)ethanamine (0.79 mL, 5.4 mmol) and DIPEA (0.16 mL, 0.9 mmol) were dissolved in DMF (1 mL) and heated by microwave irradiation at 125° C. for 10 min. The crude product was purified using automated preparative HPLC to yield N-((4-methoxy-3,5-dimethylpyridin-2-yl)methyl)-2-(4-methoxyphenyl)ethanamine as a pale yellow solid (130 mg, 48%).

A suspension of the above amine and 4,6-dichloro-pyrimidine-2-amine (71 mg, 0.43 mmol) and Et₃N (0.12 mL, 0.86 mmol) were dissolved in DMF (0.7 mL) and heated at 65° C. for 2.5 h. The crude product was purified using automated preparative HPLC to yield the desired product as a white amorphous solid (57 mg, 31%). ¹H NMR (400 MHz, CDCl₃): δ 8.16 (s, 1H), 7.02 (d, J=8.2 Hz, 1H), 6.78 (d, J=8.2 Hz, 1H), 5.89 (s, 1H), 4.89 (s, 2H), 3.75 (s, 3H), 3.72 (s, 3H), 3.55-3.48 (m, 2H), 2.73-2.67 (m, 2H), 2.21 (s, 3H), 2.13 (s, 3H). ¹³C NMR (100 MHz, CDCl₃): δ 164.0, 163.2, 161.9, 159.8, 158.2, 149.0, 129.6, 125.4, 114.1, 113.9, 92.6, 59.9, 55.2, 49.7, 32.3, 13.2, 10.7. LRMS calculated for C₂₂H₂₆ClN₅O₂ [M+H]⁺: 427.9; found 428.05.

### Example 2

### 6-Chloro-N4-((4-methoxy-3,5-dimethylpyridin-2-yl)methyl)-N4-(3-methoxyphenethyl)pyrimidine-2,4-diamine

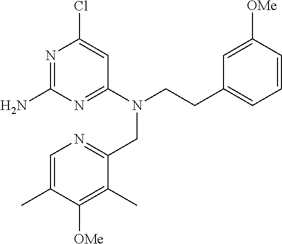 [SMILES: COc1cccc(CCN(Cc2ncc(C)c(OC)c2C)c2cc(Cl)nc(N)n2)c1]

6-Chloro-N4-((4-methoxy-3,5-dimethylpyridin-2-yl)methyl)-N4-(3-methoxyphenethyl)pyrimidine-2,4-diamine compounds was synthesized using procedure described in Example 1 and appropriate starting materials. The compound was produced as an amorphous white solid (102 mg, 31%). ¹H NMR (400 MHz, CDCl₃): δ 8.16 (s, 1H), 7.16 (dd, J=7.3 Hz, 1H), 6.73-6.69 (m, 2H), 6.65 (s, 1H), 5.90 (s, 1H), 4.95 (s, 2H), 3.77 (s, 3H), 3.72 (s, 3H), 3.58-3.53 (m, 2H), 2.78-2.70 (m, 2H), 2.19 (s, 3H), 2.13 (s, 3H). ¹³C NMR (100 MHz, CDCl₃): δ 163.9, 162.0, 159.8, 159.7, 149.1, 129.5, 125.3, 121.1, 114.5, 111.6, 109.9, 92.5, 59.9, 55.1, 49.4, 33.3, 13.2, 10.7. LRMS calculated for C₂₂H₂₆ClN₅O₂ [M+H]⁺: 427.9; found 428.05.

### Example 3

### N4-Allyl-6-chloro-N4-((4-methoxy-3,5-dimethylpyridin-2-yl)methyl)pyrimidine-2,4-diamine

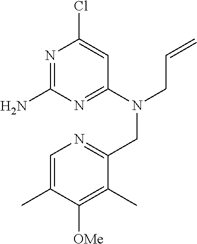 [SMILES: C=CCN(Cc1ncc(C)c(OC)c1C)c1cc(Cl)nc(N)n1]

N4-Allyl-6-chloro-N4-((4-methoxy-3,5-dimethylpyridin-2-yl)methyl)pyrimidine-2,4-diamine was synthesized using procedure described in Example 1 and appropriate starting materials. The compound was produced as an amorphous white solid (100 mg, 52%). ¹NMR (400 MHz, CDCl₃): δ 8.13 (s, 1H), 5.83 (s, 1H), 5.71-5.60 (m, 1H), 5.11-4.99 (m, 4H), 3.99 (bs, 2H), 3.70 (s, 3H), 2.18 (s, 3H), 2.16 (s, 3H). ¹³C NMR (100 MHz, CDCl₃): δ 163.8, 163.6, 161.9, 159.7, 149.0, 132.2, 125.1, 124.7, 116.6, 92.5, 59.8, 49.7, 49.6, 13.1, 10.6. LRMS calculated for C₁₆H₂₀ClN₅O [M+H]⁺: 333.8; found 334.0.

### Example 4

### 6-Chloro-N4-((4-methoxy-3,5-dimethylpyridin-2-yl)methyl)-N4-phenethylpyrimidine-2,4-diamine

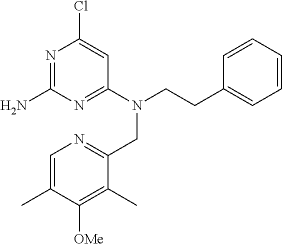 [SMILES: COc1c(C)cnc(CN(CCc2ccccc2)c2cc(Cl)nc(N)n2)c1C]

6-Chloro-N4-((4-methoxy-3,5-dimethylpyridin-2-yl)methyl)-N4-phenethylpyrimidine-2,4-diamine was synthesized using procedure described in Example 1 and appropriate starting materials. The compound was produced as an amorphous white solid (130 mg, 56%). ¹H NMR (400 MHz, CDCl₃): δ 8.19 (s, 1H), 7.29-7.13 (m, 5H), 5.93 (s, 1H), 4.84 (s, 2H), 3.74 (s, 3H), 3.70-3.51 (m, 2H), 2.81-2.79 (m, 2H), 2.23 (s, 3H), 2.15 (s, 3H). ¹³C NMR (100 MHz, CDCl₃): δ 163.9, 163.3, 162.0, 159.8, 154.1, 149.1, 138.1, 128.7, 128.5, 126.4, 125.3, 124.8, 92.4, 59.8, 49.5, 33.2, 13.2, 10.6. LRMS calculated for C₂₁H₂₄ClN₅O [M+H]⁺: 397.9; found 398.0.

### Example 5

### 6-Chloro-N4-((4-methoxy-3,5-dimethylpyridin-2-yl)methyl)-N4-propylpyrimidine-2,4-diamine

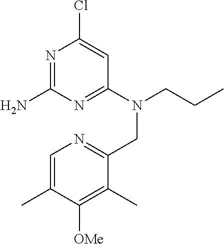 [SMILES: CCCN(Cc1ncc(C)c(OC)c1C)c1cc(Cl)nc(N)n1]

6-Chloro-N4-((4-methoxy-3,5-dimethylpyridin-2-yl)methyl)-N4-propylpyrimidine-2,4-diamine was synthesized using procedure described in Example 1 and appropriate starting materials. The compound was produced as an amorphous white solid (85 mg, 45%). ¹H NMR (400 MHz, CDCl₃): δ 7.95 (s, 1H), 5.65 (s, 1H), 5.00 (s, 2H), 3.54 (s, 3H), 3.12-2.90 (m, 2H), 2.02 (s, 3H), 1.98 (s, 3H), 1.32-1.28 (m, 2H), 0.66-0.61 (m, 3H). ¹³C NMR (100 MHz, CDCl₃): δ 163.4, 161.7, 161.3, 159.0, 148.5, 124.7, 124.3, 108.5, 91.6, 59.4, 49.7, 48.8, 19.6, 12.7, 10.8, 10.2. LRMS calculated for C₁₆H₂₂ClN₅O [M+H]⁺: 335.8; found 336.0.

### Example 6

### 6-Chloro-N4-((4-methoxy-3,5-dimethylpyridin-2-yl)methyl)-N4-(2-morpholinoethyl)pyrimidine-2,4-diamine

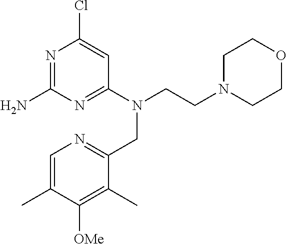 [SMILES: COc1c(C)cnc(CN(CCN2CCOCC2)c2cc(Cl)nc(N)n2)c1C]

6-Chloro-N4-((4-methoxy-3,5-dimethylpyridin-2-yl)methyl)-N4-(2-morpholinoethyl)pyrimidine-2,4-diamine was synthesized using procedure described in Example 1 and appropriate starting materials. The compound was produced as an amorphous yellow solid (130 mg, 52%). ¹H NMR (400 MHz, CDCl₃): δ 8.11 (s, 1H), 5.84 (s, 1H), 5.17 (s, 1H), 3.74 (s, 3H), 3.65-3.62 (m, 4H), 3.54-3.50 (m, 2H), 2.44 (bs, 6H), 2.18 (s, 3H), 2.14 (s, 3H). ¹³C NMR (100 MHz, CDCl₃): δ 163.9, 163.4, 161.9, 159.6, 148.9, 125.3, 92.3, 66.4, 59.8, 55.2, 53.5, 44.5, 13.2, 10.6. LRMS calculated for C₁₉H₂₇ClN₆O₂ [M+H]⁺: 406.9; found 407.0.

### Example 7

### 6-Chloro-N4-ethyl-N4-((4-methoxy-3,5-dimethylpyridin-2-yl)methyl)pyrimidine-2,4-diamine

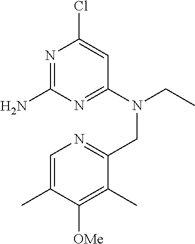 [SMILES: CCN(Cc1ncc(C)c(OC)c1C)c1cc(Cl)nc(N)n1]

6-Chloro-N4-ethyl-N4-((4-methoxy-3,5-dimethylpyridin-2-yl)methyl)pyrimidine-2,4-diamine was synthesized using procedure described in Example 1 and appropriate starting materials. The compound was produced as an amorphous yellow solid (71 mg, 67%). ¹H NMR (400 MHz, CDCl₃): δ 8.15 (s, 1H), 5.88 (s, 1H), 4.93 (s, 2H), 3.71 (s, 3H), 3.37 (bs, 2H), 2.20 (s, 3H), 2.16 (s, 3H), 1.02-0.98 (m, 3H). ¹³C NMR (100 MHz, CDCl₃): δ 163.9, 163.0, 162.0, 159.8, 155.0, 149.0, 125.3, 125.0, 92.3, 59.8, 49.7, 41.8, 13.2, 11.8, 10.7. LRMS calculated for C₁₅H₂₀ClN₅O [M+H]⁺: 321.8; found 322.0.

### Example 8

### N4-butyl-6-chloro-N4-((4-methoxy-3,5-dimethylpyridin-2-yl)methyl)pyrimidine-2,4-diamine

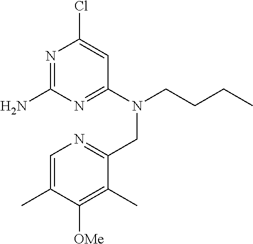 [SMILES: CCCCN(Cc1ncc(C)c(OC)c1C)c1cc(Cl)nc(N)n1]

N4-butyl-6-chloro-N4-((4-methoxy-3,5-dimethylpyridin-2-yl)methyl)pyrimidine-2,4-diamine was synthesized using procedure described in Example 1 and appropriate starting materials. The compound was produced as an amorphous white solid (106 mg, 45%). ¹H NMR (400 MHz, CDCl₃): δ 8.11 (s, 1H), 5.81 (s, 1H), 5.13 (s, 2H), 3.68 (s, 3H), 3.26 (bs, 2H), 2.17 (s, 3H), 2.12 (s, 3H), 1.41-1.38 (m, 2H), 1.23-1.18 (m, 2H), 0.84-0.80 (m, 3H). ¹³C NMR (100 MHz, CDCl₃): δ 163.8, 163.2, 161.9, 159.5, 154.1, 148.9, 125.1, 124.8, 92.1, 59.7, 50.1, 47.2, 28.7, 19.9, 13.7, 13.1, 10.6. LRMS calculated for C₁₇H₂₄ClN₅O [M+H]⁺: 349.8; found 350.0.

### Example 9

### 6-Chloro-N4-hexyl-N4-((4-methoxy-3,5-dimethylpyridin-2-yl)methyl)pyrimidine-2,4-diamine

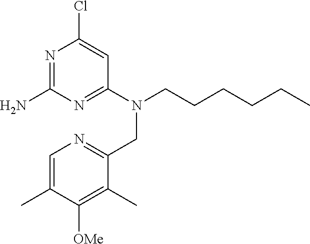 [SMILES: CCCCCCN(Cc1ncc(C)c(OC)c1C)c1cc(Cl)nc(N)n1]

6-Chloro-N4-hexyl-N4-((4-methoxy-3,5-dimethylpyridin-2-yl)methyl)pyrimidine-2,4-diamine was synthesized using procedure described in Example 1 and appropriate starting materials. The compound was produced as an amorphous white solid (59 mg, 37%). ¹H NMR (400 MHz, CDCl₃): δ 8.14 (s, 1H), 5.84 (s, 1H), 4.95 (s, 2H), 3.71 (s, 3H), 3.28 (bs, 2H), 2.22 (s, 3H), 2.17 (s, 3H), 1.48-1.40 (m, 2H), 1.27-1.17 (m, 6H), 0.86-0.82 (m, 3H). ¹³C NMR (100 MHz, CDCl₃): δ 163.9, 163.2, 161.9, 159.6, 154.4, 149.0, 125.2, 124.9, 92.3, 59.8, 50.2, 47.5, 31.4, 26.6, 26.4, 22.4, 13.9, 13.2, 10.7. LRMS calculated for C₁₉H₂₈ClN₅O [M+H]⁺: 377.9; found 378.0.

### Example 10

### 6-Chloro-N4-(4-chlorophenethyl)-N4-((4-methoxy-3,5-dimethylpyridin-2-yl)methyl)pyrimidine-2,4-diamine

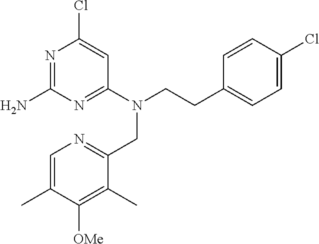 [SMILES: COc1c(C)cnc(CN(CCc2ccc(Cl)cc2)c2cc(Cl)nc(N)n2)c1C]

6-Chloro-N4-(4-chlorophenethyl)-N4-((4-methoxy-3,5-dimethylpyridin-2-yl)methyl)pyrimidine-2,4-diamine was synthesized using procedure described in Example 1 and appropriate starting materials. The compound was produced as an amorphous white solid (105 mg, 38%). ¹H NMR (400 MHz, CDCl₃): δ 8.17 (s, 1H), 7.22-7.19 (m, 2H), 7.04 (dd, J=8.2 Hz, J=2.3 Hz, 2H), 5.87 (s, 1H), 5.04 (s, 2H), 3.73 (s, 3H), 3.58 (bs, 2H), 2.77-2.73 (m, 2H), 2.21 (s, 3H), 2.13 (s, 3H). ¹³C NMR (100 MHz, CDCl₃): δ 164.5, 163.2, 161.9, 159.9, 154.1, 148.4, 137.2, 132.2,130.1, 128.6, 125.6, 125.1, 92.5, 59.9, 50.2, 49.6, 32.7, 13.3, 10.7. LRMS calculated for C₂₁H₂₃Cl₂N₅O [M+H]⁺: 432.3; found 432.0.

### Example 11

### N4-(2-(Benzo[d][1,3]dioxol-5-yl)ethyl)-6-chloro-N4-((4-methoxy-3,5-dimethylpyridin-2-yl)methyl)pyrimidine-2,4-diamine

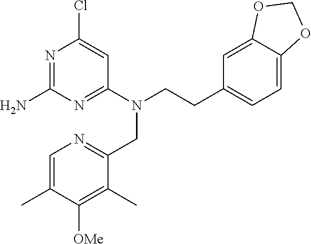 [SMILES: COc1c(C)cnc(CN(CCc2ccc3c(c2)OCO3)c2cc(Cl)nc(N)n2)c1C]

N4-(2-(Benzo[d][1,3]dioxol-5-yl)ethyl)-6-chloro-N4-((4-methoxy-3,5-dimethylpyridin-2-yl)methyl)pyrimidine-2,4-diamine was synthesized using procedure described in Example 1 and appropriate starting materials. The compound was produced as an amorphous yellow solid (10 mg, 26%). ¹H NMR (400 MHz, CDCl₃): δ 8.19 (s, 1H), 6.77-6.57 (m, 3H), 5.94 (s, 1H), 5.92 (s, 1H), 4.80 (s, 2H), 3.75 (s, 3H), 3.56-3.52 (m, 2H), 2.78-2.69 (m, 2H), 2.24 (s, 3H), 2.17 (s, 3H). ¹³C NMR (100 MHz, CDCl₃): δ 164.0, 161.9, 161.0, 159.9, 149.2, 147.7, 146.1, 125.4, 121.6, 109.1, 108.3, 100.9, 92.6, 59.9, 49.2, 39.3, 33.0, 13.3, 10.7. LRMS calculated for C₂₂H₂₄ClN₅O₃ [M+H]⁺: 441.9; found 442.0.

### Example 12

### 6-Chloro-N4-((4-methoxy-3,5-dimethylpyridin-2-yl)methyl)-N4-methylpyrimidine-2,4-diamine

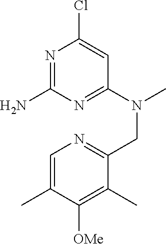 [SMILES: COc1c(C)cnc(CN(C)c2cc(Cl)nc(N)n2)c1C]

6-Chloro-N4-((4-methoxy-3,5-dimethylpyridin-2-yl)methyl)-N4-methylpyrimidine-2,4-diamine was synthesized using procedure described in Example 1 and appropriate starting materials. The compound was produced as an amorphous yellow solid (300 mg, 60%). ¹H NMR (400 MHz, DMSO d₆): δ 8.06 (s, 1H), 6.32 (s, 2H), 5.89 (s, 1H), 4.73 (s, 2H), 3.67 (s, 3H), 2.90 (s, 3H), 2.12 (s, 3H), 2.11 (s, 3H). ¹³C NMR (100 MHz, DMSO d₆): δ 163.9, 163.3, 162.3, 158.9, 154.1, 148.5, 124.5, 123.9, 90.6, 59.8, 50.1, 36.0, 12.9, 10.2. LRMS calculated for C₁₄H₁₈ClN₅O [M+H]⁺: 307.8; found 308.0.

### Example 13

### 13 3-((2-Amino-6-chloropyrimidin-4-yl)((4-methoxy-3,5-dimethylpyridin-2-yl)methyl)amino)propan-1-ol

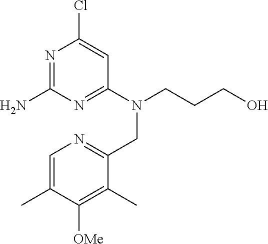 [SMILES: COc1c(C)cnc(CN(CCCO)c2cc(Cl)nc(N)n2)c1C]

13 3-((2-Amino-6-chloropyrimidin-4-yl)((4-methoxy-3,5-dimethylpyridin-2-yl)methyl)amino)propan-1-ol was synthesized using procedure described in Example 1 and appropriate starting materials. The compound was produced as an amorphous white solid (120 mg, 48%). ¹H NMR (400 MHz, DMSO d₆): δ 8.06 (s, 1H), 6.30 (s, 2H), 5.90 (s, 1H), 3.67 (s, 3H), 3.36 (t, J=5.9 Hz, 4H), 2.13 (s, 3H), 2.12 (s, 3H), 1.62-1.52 (m, 2H). ¹³C NMR (100 MHz, DMSO d₆): δ 163.3, 162.4, 158.9, 148.5, 124.6, 123.9, 90.7, 59.8, 58.3, 49.2, 45.0, 30.1, 12.9, 10.2. LRMS calculated for C₁₆H₂₂ClN₅O₂ [M+H]⁺: 351.8; found 352.0.

### Example 14

### N4-(3-Bromophenethyl)-6-chloro-N4-((4-methoxy-3,5-dimethylpyridin-2-yl)methyl)pyrimidine-2,4-diamine

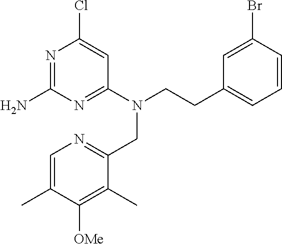 [SMILES: COc1c(C)cnc(CN(CCc2cccc(Br)c2)c2cc(Cl)nc(N)n2)c1C]

N4-(3-Bromophenethyl)-6-chloro-N4-((4-methoxy-3,5-dimethylpyridin-2-yl)methyl)pyrimidine-2,4-diamine was synthesized using procedure described in Example 1 and appropriate starting materials. The compound was produced as an amorphous white solid (146 mg, 36%). ¹H NMR (400 MHz, DMSO d₆): δ 8.10 (s, 1H), 7.44 (s, 1H), 7.37-7.35 (d, J=6.4 Hz, 1H), 7.40-7.21 (m, 2H), 6.43 (s, 2H), 5.93 (s, 1H), 3.69 (s, 3H), 3.59-3.44 (m, 2H), 2.82-2.75 (m, 2H), 2.15 (s, 3H), 2.14 (s, 3H). ¹³C NMR (100 MHz, DMSO d₆): δ 163.2, 162.4, 158.8, 148.4, 142.1, 131.5, 130.3, 128.9, 127.9, 124.5, 123.9, 121.6, 90.8, 59.7, 49.1, 32.0, 12.8, 10.1. LRMS calculated for C₂₁H₂₃BrClN₅O [M+H]⁺: 476.0, 478.0; found 476.0, 478.0.

### Example 15

### N4-Benzyl-6-chloro-N4-((4-methoxy-3,5-dimethylpyridin-2-yl)methyl)pyrimidine-2,4-diamine

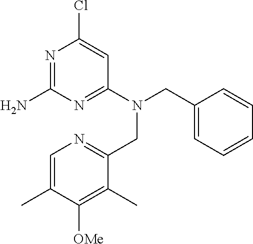 [SMILES: COc1c(C)cnc(CN(Cc2ccccc2)c2cc(Cl)nc(N)n2)c1C]

N4-Benzyl-6-chloro-N4-((4-methoxy-3,5-dimethylpyridin-2-y0methyl)pyrimidine-2,4-diamine was synthesized using procedure described in Example 1 and appropriate starting materials. The compound was produced as an amorphous white solid (10 mg, 23%). ¹H NMR (400 MHz, DMSO d₆): δ 8.13 (s, 1H), 7.32-7.19 (m, 5H), 6.45 (s, 2H), 5.90 (s, 1H), 4.69 (s, 2H), 3.68 (s, 3H), 2.16 (s, 3H), 2.11 (s, 3H). ¹³C NMR (100 MHz, DMSO d₆): δ 164.0, 163.2, 162.4, 158.9, 148.5, 138.0, 128.4, 127.1, 126.8, 124.6, 124.0, 90.8, 59.7, 49.3, 12.8, 10.1. LRMS calculated for C₂₀H₂₂ClN₅O [M+H]⁺: 383.8; found 384.0.

### Example 16

### 6-Chloro-N4-isopropyl-N4-((4-methoxy-3,5-dimethylpyridin-2-yl)methyl)pyrimidine-2,4-diamine

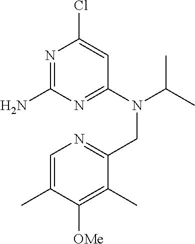 [SMILES: COc1c(C)cnc(CN(c2cc(Cl)nc(N)n2)C(C)C)c1C]

6-Chloro-N4-isopropyl-N4-((4-methoxy-3,5-dimethylpyridin-2-yl)methyl)pyrimidine-2,4-diamine was synthesized using procedure described in Example 1 and appropriate starting materials. The compound was produced as an amorphous white solid (96 mg, 40%). ¹H NMR (400 MHz, CDCl₃): δ 8.14 (s, 1H), 5.71 (s, 1H), 4.69 (s, 2H), 3.75 (s, 3H), 2.23 (s, 3H), 2.22 (s, 3H), 1.84 (s, 1H), 1.14 (s, 3H), 1.13 (s, 3H). LRMS calculated for C₁₆H₂₂ClN₅O [M+H]⁺: 335.8; found 336.0.

### Example 17

### 6-Chloro-N4-((4-chloro-3,5-dimethylpyridin-2-yl)methyl)-N4-(4-chlorophenethyl)pyrimidine-2,4-diamine

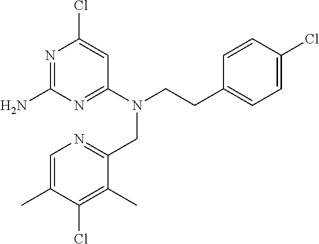 [SMILES: Cc1cnc(CN(CCc2ccc(Cl)cc2)c2cc(Cl)nc(N)n2)c(C)c1Cl]

6-Chloro-N4-((4-chloro-3,5-dimethylpyridin-2-yl)methyl)-N4-(4-chlorophenethyl)pyrimidine-2,4-diamine was synthesized using procedure described in Example 1 and appropriate starting materials. The compound was produced as an amorphous white solid (104 mg, 38%). ¹H NMR (400 MHz, DMSO d₆): δ 8.17 (s, 1H), 7.26-7.19 (m, 4H), 6.41 (s, 2H), 5.88 (1H), 3.57 (s, 2H), 2.79-2.73 (m, 2H), 2.26 (s, 3H), 2.19 (s, 3H). ¹³C NMR (100 MHz, DMSO d₆): δ 163.1, 162.4, 158.9, 154.0, 147.1, 143.5, 138.1, 130.8, 130.6, 129.8, 128.9, 128.1, 90.8, 49.3, 32.0, 16.8, 14.3. LRMS calculated for C₂₀H₂₀Cl₃N₅ [M+H]⁺: 436.0, 438.0; found 436.0, 438.0.

### Example 18

### 6-Chloro-N4-((4-chloro-3,5-dimethylpyridin-2-yl)methyl)-N4-methylpyrimidine-2,4-diamine

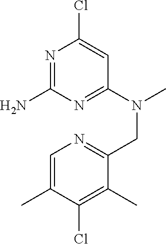 [SMILES: Cc1cnc(CN(C)c2cc(Cl)nc(N)n2)c(C)c1Cl]

6-Chloro-N4-((4-chloro-3,5-dimethylpyridin-2-yl)methyl)-N4-methylpyrimidine-2,4-diamine was synthesized using procedure described in Example 1 and appropriate starting materials. The compound was produced as an amorphous yellow solid (4.1 mg, 26%). ¹H NMR (400 MHz, CDCl₃): δ 8.13 (s, 1H), 5.85 (s, 1H), 4.70 (s, 2H), 2.88 (s, 3H), 2.27 (s, 3H), 2.25 (s, 3H).). ¹³C NMR (100 MHz, CDCl₃): δ 163.8, 161.8, 160.0, 147.5, 145.0, 130.8, 130.2, 92.4, 52.0, 35.4, 17.4, 14.9. LRMS calculated for C₁₃H₁₅Cl₂N₅ [M+H]⁺: 312.2; found 312.0.

### Example 19

### N4-((4-Bromo-3,5-dimethylpyridin-2-yl)methyl)-6-chloro-N4-methylpyrimidine-2,4-diamine

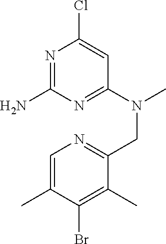 [SMILES: Cc1cnc(CN(C)c2cc(Cl)nc(N)n2)c(C)c1Br]

N4-((4-Bromo-3,5-dimethylpyridin-2-yl)methyl)-6-chloro-N4-methylpyrimidine-2,4-diamine was synthesized using procedure described in Example 1 and appropriate starting materials. The compound was produced as an amorphous yellow solid (102 mg, 45%). ¹H NMR (400 MHz, CDCl₃): δ 8.16 (s, 1H), 5.93 (s, 1H), 4.89 (s, 2H), 2.95 (s, 3H), 2.38 (s, 3H), 2.35 (s, 3H). LRMS calculated for C₁₃H₁₅BrClN₅ [M+H]⁺: 356.0, 358.0; found 356.0, 358.0.

### Example 20

### 2-((4-Hydroxy-3,5-dimethylpyridin-2-yl)methyl)isoindoline-1,3-dione

A suspension of 2-(chloromethyl)-4-methoxy-3,5-dimethylpyridine hydrochloride (10 g, 45 mmol), pthalimide (7.28 g, 49.5 mmol) and K₂CO₃ (25 g, 180.8 mmol) were dissolved in DMF (200 mL) and reacted at room temperature for 16 h. To the white solid formed saturated NaHCO₃ was added until basic and filtered to obtain 2-((4-methoxy-3,5-dimethylpyridin-2-yl)methyl)isoindoline-1,3-dione as a white solid in quantitative yield. ¹H NMR (400 MHz, CDCl₃): δ 8.04 (s, 1H), 7.89-7.87 (m, 2H), 7.73-7.72 (m, 2H), 4.92 (s, 2H), 3.75 (s, 3H), 2.32 (s, 3H), 2.18 (s, 3H).

To a solution of the above product (737 mg, 2.49 mmol) in THF/EtOH (5 mL/5 mL) was added conc. HCl (0.41 mL) and it was concentrated. The residue was dissolved in DMF (4 mL) and NaBr (256 mg, 2.49 mmol) was added and heated at 120° C. for 1 h. To this solution EtOAc was added and the precipitate was filtered to obtain 2-((4-hydroxy-3,5-dimethylpyridin-2-yl)methyl)isoindoline-1,3-dione as a white solid in quantitative yield. LCMS (ESI) calculated for C₁₆H₁₄N₂O₃ [M+H]⁺: 282.3; found, 283.0.

### Example 21

### 6-Chloro-N4-((4-chloro-3,5-dimethylpyridin-2-yl)methyl)pyrimidine-2,4-diamine

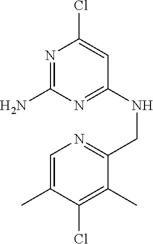 [SMILES: Cc1cnc(CNc2cc(Cl)nc(N)n2)c(C)c1Cl]

2-((4-Hydroxy-3,5-dimethylpyridin-2-yl)methyl)isoindoline-1,3-dione (50 mg, 0.17 mmol), synthesized as shown above in Example 20, and POCl₃ (0.15 mL, 1.66 mmol) were combined in a sealed tube and heated at 110° C. for 45 min The solution was cooled, added to ice water and basified with 40% KOH. The formed precipitate was filtered to obtain 2-((4-chloro-3,5-dimethylpyridin-2-yl)methyl)isoindoline-1,3-dione as a white solid (22.9 mg, 45%). LC-MS (ESI) calculated for C₁₆H₁₃ClN₂O₂ [M+1]⁺: 300.7; found, 301.0.

To a solution of 2-((4-chloro-3,5-dimethylpyridin-2-yl)methyl)isoindoline-1,3-dione (22.9 mg, 0.076 mmol), in EtOH/Toluene (0.4 mL: 0.2 mL) was added NH₂NH₂.H₂O (22 μL, 0.456 mmol) and heated at 90° C. for 20 min The reaction mixture was cooled, filtered, and the filtrate was concentrated and washed with CH₂Cl₂ to obtain the crude amine as a yellow oil which was directly used for the next step without further purification. LC-MS (ESI) calculated for C₈H₁₁ClN₂ [M+1]⁺: 170.6; found, 171.0.

A suspension of 4,6-dichloropyrimidin-2-amine (11.5 mg, 0.07 mmol), DIPEA (24 μL, 0.14 mmol) and (4-chloro-3,5-dimethylpyridin-2-yl)methanamine (12 mg, 0.07 mmol) in EtOH (0.5 mL) was heated by microwave irradiation at 160° C. for 10 min The crude product was purified using automated preparative HPLC to yield the desired product as a white amorphous solid (10 mg, 44% over 2 steps). ¹H NMR (400 MHz, DMSO d₆): δ 8.25 (s, 1H), 6.37 (bs, 2H), 5.89 (s, 1H), 4.51 (s, 2H), 2.29 (s, 3H), 2.24 (s, 3H). ¹³C NMR (100 MHz, DMSO d₆): δ 163.9, 162.9, 157.1, 154.6, 146.9, 143.7, 130.2, 129.0, 93.1, 44.1, 16.9, 14.6. LRMS calculated for C₁₂H₁₃Cl₂N₅ [M+H]⁺: 298.2; found 298.0.

### Example 22

### Synthesis of 2-((1,3-dioxoisoindolin-2-yl)methyl)-3,5-dimethylpyridin-4-yl trifluoromethanesulfonate

To a solution of 2-((4-hydroxy-3,5-dimethylpyridin-2-yl)methyl)isoindoline-1,3-dione (3.2 g, 11.3 mmol), synthesized as shown above in Example 20, and DIPEA (2.36 mL, 13.6 mmol) in CH₂Cl₂ (98 mL) cooled to 0° C., trifluoromethane sulfonic anhydride (2.29 mL, 13.6 mmol) was added dropwise. The reaction mixture was cooled, water added, and extraction performed with CH₂Cl₂. The organic layer was dried over Na₂SO₄, filtered and evaporated solvent in vacuo to obtain crude product as an orange solid (4.0 g, 85%) which was used for the next step without further purification. LRMS calculated for C₁₇H₁₃F₃N₂O₅S [M+H]⁺: 414.4; found 415.0.

### Example 23

### N4-((4-Bromo-3,5-dimethylpyridin-2-yl)methyl)-6-chloropyrimidine-2,4-diamine

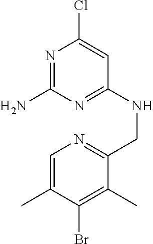 [SMILES: Cc1cnc(CNc2cc(Cl)nc(N)n2)c(C)c1Br]

To a solution in THF/EtOH (15 mL/15 mL) of 2-((1,3-dioxoisoindolin-2-yl)methyl)-3,5-dimethylpyridin-4-yl trifluoromethanesulfonate (3.19 g, 7.69 mmol), synthesized as shown in Example 22 above in THF/EtOH (15 mL/15 mL) was added 47% HBr in H₂O (0.84 mL, 15.4 mmol) and concentrated. The residue was dissolved in DMF and NaBr (1.58 g, 15.4 mmol) was added and heated at 110° C. for 1.5 h. It was then poured into water and extracted with EtOAc. The organic layer was dried over Na₂SO₄, filtered and evaporated solvent in vacuo to obtain crude product as a pale yellow solid (2.0 g, 75%). LRMS calculated for C₁₆H₁₃BrN₂O₂ [M+H]⁺: 345.2 found 345.0.

To a solution of 2-((4-bromo-3,5-dimethylpyridin-2-yl)methyl)isoindoline-1,3-dione (2 g, 5.8 mmol) in EtOH/Toluene (30 mL: 15 mL) was added NH₂NH₂.H₂O (1.4 mL, 28.9 mmol) and heated at 110° C. for 20 min. The reaction mixture was cooled, filtered, and the filtrate was concentrated. Added 2M NaOH and extracted with CH₂Cl₂ to obtain the crude amine as a brown oil (542 mg, 44%). LC-MS (ESI) cald for C₈H₁₁BrN₂ [M+1]⁺, 215.1; found 215.0.

A suspension of 4,6-dichloropyrimidin-2-amine (411.5 mg, 2.51 mmol), DIPEA (2.45 mL, 14.1 mmol) and (4-bromo-3,5-dimethylpyridin-2-yl)methanamine (542 mg, 2.51 mmol) in nBuOH (12 mL) were heated at 115° C. for 1 h. The crude product was purified using automated preparative HPLC to yield the desired product as a white amorphous solid (400 mg, 47%). ¹H NMR (400 MHz, DMSO d₆): δ 8.20 (s, 1H), 6.38 (s, 2H), 5.89 (s, 1H), 4.53 (s, 2H), 2.33 (s, 3H), 2.26 (s, 3H). ¹³C NMR (100 MHz, DMSO d₆): δ 163.8, 162.9, 154.2, 146.6, 137.9, 132.4, 131.1, 93.1, 44.0, 20.1, 17.8. LRMS calculated for C₁₂H₁₃BrClN₅ [M+H]⁺: 342.6; found 343.8.

### Example 24

### 6-Chloro-N4-((4-iodo-3,5-dimethylpyridin-2-yl)methyl)pyrimidine-2,4-diamine

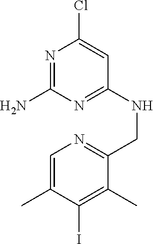 [SMILES: Cc1cnc(CNc2cc(Cl)nc(N)n2)c(C)c1I]

6-Chloro-N4-((4-iodo-3,5-dimethylpyridin-2-yl)methyl)pyrimidine-2,4-diamine was synthesized following the methods of Examples 20-23 using appropriate starting material. The compound was produced as a white amorphous solid (31 mg, 47%). ¹H NMR (400 MHz, CDCl₃): δ 8.12 (s, 1H), 6.72 (bs, 1H), 5.98 (s, 1H), 4.85 (s, 2H), 4.52 (s, 2H), 2.48 (s, 3H), 2.44 (s, 3H). LRMS calculated for C₁₂H₁₃ClIN₅ [M+H]⁺: 389.6; found 390.0.

### Example 25

### 2-Bis-(tert-butoxycarbonyl)amino-6-chloro-N4-((4-methoxy-3,5-dimethylpyridin-2-yl)methyl)pyrimidine-4-amine

To a solution of 4,6-dichloropyrimidin-2-amine (2.2 g, 13.4 mmol), DMAP (164 mg, 1.34 mmol) in THF (20 mL) and Boc₂O (6.4 g, 29.3 mmol) was added, and the mixture stirred at room temperature overnight. The solvent was evaporated under reduced pressure and the resulting residue was purified by column chromatography (silica gel, EtOAc/hexanes) to obtain 2-Bis-(tert-butoxycarbonyl)amino-4,6-dichloropyrimidine as a white solid in quantitative yield.

¹H NMR (400 MHz, DMSO d₆): δ 8.02 (s, 1H), 1.40 (s, 18H).

A suspension of the Boc protected pyrimidine (460 mg, 1.26 mmol) synthesized above, DIPEA (1.23 mL, 7.06 mmol) and (4-methoxy-3,5-dimethylpyridin-2-yl)methanamine (211 mg, 1.26 mmol) in nBuOH (6 mL) were heated at 115° C. for 1 h. The mixture was cooled and solvent was evaporated in vacuo. The resulted residue was dissolved in water and extracted with CH₂Cl₂and EtOAc. The organic layer was dried over Na₂SO₄, filtered and solvent evaporated in vacuo to obtain product as a pale yellow oil (523.6 mg, 84%).

### Example 26

### N-(2-amino-6-chloropyrimidin-4-yl)-N-((4-methoxy-3,5-dimethylpyridin-2-yl)methyl)-3,3-dimethylbutanamide

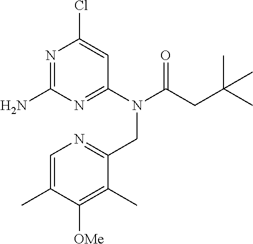 [SMILES: COc1c(C)cnc(CN(C(=O)CC(C)(C)C)c2cc(Cl)nc(N)n2)c1C]

2-Bis-(tert-butoxycarbonyl)amino-6-chloro-N4-((4-methoxy-3,5-dimethylpyridin-2-yl)methyl)pyrimidine-4-amine (120 mg, 0.24 mmol), synthesized as shown above in Example 26, was dissolved in anhydrous CH₂Cl₂ (2.5 mL), cooled to 0° C., and DIPEA (0.12 mL, 0.72 mmol) was added drop wise followed by 3,3-dimethylbutanoyl chloride (67 μL, 0.48 mmol). The reaction mixture was allowed to warm up to room temperature and stirred for 16 h. After concentration under reduced pressure the residue was dissolved in anhydrous CH₂Cl₂ (1.4 mL), TFA (0.48 mL) was added and the resulting solution stirred at room temperature for 30 min. The crude product obtained was purified using automated preparative HPLC to yield the desired product as a yellow amorphous solid (52 mg, 55%). ¹H NMR (400 MHz, CDCl₃): δ 8.64 (s, 1H), 6.61 (s, 1H), 5.31 (s, 2H), 3.94 (s, 3H), 2.52 (s, 2H), 2.35 (s, 3H), 2.33 (s, 3H), 0.97 (9H). LRMS calculated for C₁₉H₂₆ClN₅O₂ [M+H]⁺: 391.8; found 392.0.

### Example 27

### N-(2-Amino-6-chloropyrimidin-4-yl)-4-chloro-N-((4-methoxy-3,5-dimethylpyridin-2-yl)methyl)butanamide

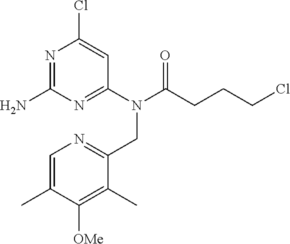 [SMILES: COc1c(C)cnc(CN(C(=O)CCCCl)c2cc(Cl)nc(N)n2)c1C]

N-(2-Amino-6-chloropyrimidin-4-yl)-4-chloro-N-((4-methoxy-3,5-dimethylpyridin-2-yl)methyl)butanamide was synthesized using the procedure for shown in example 26 in a similar by using appropriate starting material. The compound was obtained as a white amorphous solid (20 mg, 42%). ¹H NMR (400 MHz, CDCl₃): δ 8.52 (s, 1H), 6.78 (s, 1H), 5.26 (s, 2H), 3.91 (s, 3H), 3.63-3.58 (m, 2H), 2.84-2.80 (m, 2H), 2.32 (s, 3H), 2.30 (s, 3H), 2.17-2.12 (m, 2H). LRMS calculated for C₁₇H₂₁Cl₂N₅O₂ [M+H]⁺: 398.3; found 398.0.

### Example 28

### Materials and Methods for Determining Structure Activity Relationships of Heat Shock Protein 90 (Hsp90) Inhibitors

Hydroxyethyl piperazineethanesulfonic acid (HEPES), potassium chloride (KCl), magnesium chloride (MgCl₂), sodium molybdate, dithiothreitol (DTT), tergitol Type NP-40 (NP40), dimethyl sulphoxide (DMSO), and bovine serum albumin (BSA) were purchased from Sigma (St. Louis, Mo.). Fluorescein isothiocyanate (FITC)-labeled geldanamycin (GM-FITC) (lot#3-A110334d) was purchased from Enzo Life Sciences (Farmingdale, N.Y.). The PolarStar Omega plate reader used for fluorescence polarization readings was a product of BMG-Lab Tech (Stafford, Tex.). Small molecular weight scaffolds were obtained from Sorrento Technologies. Hsp90 inhibitor compounds were designed and synthesized in the laboratory of Dr. Nick Cosford, Burnham Institute (La Jolla, Calif.).

### Example 29

### Cloning, Expression, and Purification of Full Length Human Hsp90

The structural gene encoding amino acid residues 1 to 732 of human Hsp90 (GenBank: BC121062.2) was cloned from human cDNA isolated from mixed tissue types (catalog #MHS4426-99625755; Lot #40118488; Thermo-Fisher Scientific Inc., West Palm Beach, Fla.). Cloning was accomplished using a PCR cloning kit (AccuPrime Pfx; Invitrogen Inc., Carlsbad Calif.) and a thermo-cycler (Model #DNA-Engine; Biorad Inc.; Hercules, Calif.) utilizing forward (5′-TGA CAG GAT CCT GAG GAA ACC CAG ACC-3′, SEQ ID NO:) and reverse (5′-CGC ATG GAA GAA GTA GAC TAA GGA TCC ATA TAT-3′ SEQ ID NO:) oligonucleotide primers synthesized at Integrated DNA Technologies, Inc. (Coralville, Iowa). The resulting DNA encoding the Hsp90 structural gene was then sub-cloned into an E. coli expression vector system (pET15b; EMD-Millipore Inc., Billerica, Mass.). The expression vector containing full-length Hsp90 was transformed into BL21DE3 cells (EMD-Millipore Inc., Billerica, Mass.) and cultured. The resulting expression culture was frozen at −80° C. in storage buffer containing 25% glycerol until further use.

Full-length human Hsp90 protein was produced by growing 6 liters (L) of E. coli transformed with the expression vector containing full length Hsp90 gene. Frozen cultures (stored at −80° C.) of the E. coli were used to inoculate (100 μl E. coli per 100 mL media) 250 mL culture flasks containing 100 mL of sterilized Luria-Bertani Broth supplemented with sodium ampicillin (100 μl/mL), and grown for 16 h at 37° C. under constant agitation (250 rpm) to obtain starter cultures. Starter cultures were used to inoculate (10 mL per L) six (2.8 L) fluted shaker flasks containing 1 L of sterilized Luria-Bertani Broth supplemented with sodium ampicillin (100 ug/mL) and grown at 37° C. under constant agitation (250 rpm). Culture growth was monitored by light scattering at 600 nm utilizing a micro-titer plate reader (Model #Synergy HT; BioTek Inc., Winooski, Vt.) and a round well 96 well micro-titer plate. Cultures were supplemented with Isopropyl β-D-1-thiogalactopyranoside (IPTG, 1 mM final concentration) by the addition (5 mL per L) of 200× filter-sterilized IPTG aqueous stock solution, when an optical density of 0.4 was reached, and cultures were then grown at 25° C. for 18 h. After 16 h, cultures were harvested in 1 L polycarbonate buckets using a refrigerated centrifuge (Model #DPR-6000, Damon Inc., Needham Heights, Mass.) equipped with a swinging bucket rotor (Model #981, Damon Inc., Needham Heights, Mass.) at 4,000 rpm for 15 min at 4° C. The supernatant was removed and pellets were frozen at −80° C.

### Example 30

### Determination of the K_{d} Value Fluorescein Isothiocyanate (FITC)-labeled Geldanamycin (GA-FITC)

Eighty five micro liters (μl) of a binding assay buffer (20 mM HEPES, pH 7.5, 50 mM KCl, 5 mM MgCl₂, 20 mM sodium molybdate, 0.01% NP-40, 2 mM DTT, and 0.1 mg/mL BSA) were added to a 96 well black plate placed on ice. DMSO (3 μl) was added to the wells, followed by the addition of either 10 μl human full length HSP90 (50 nM final concentration) diluted in the binding assay buffer or 10 μl of the buffer alone. The plates were incubated at 4° C. on a plate shaker for 24 hours. Two μl of 50× GA-FITC titrate within a final range of 160 nM to 0.3125 nM in DMSO were added to all wells, followed by incubation at ambient temperature for 1 h with agitation. Fluorescence polarization readings were taken at excitation and emission wavelengths of 480 and 500 nm respectively. Measurements were performed in duplicate. Specific binding was calculated by subtracting polarization values in the presence of

Hsp90 from those in the absence of Hsp90. GA-FITC K_{d} values were calculated using Prism software (GraphPad Software, Inc., San Diego, Calif.).

A 10 point concentration response curve for binding of GM-FITC to Hsp90 was obtained. The concentration of GM-FITC used ranged from 160 nM to 0.3125 nM, which produced a saturable concentration response curve. The K_{d} for GM-FITC was determined from the curve to be 3.1 nM. The value of binding maximum (Bₘₐₓ) was determined to be 188 nM.

### Example 31

### Hsp90 Binding Assay

Hsp90 binding assays was performed generally according to the procedure described in J. Biomol. Screening 9:375, 2004 and Anal. Biochem. 350:202, 2006. Hsp90 inhibitors were solubilized in DMSO at a stock concentration of 50 mM. Hsp90 inhibitors were screened at 10 μM and 1 μM, or titrated in DMSO (2-fold dilutions top achieve a final concentration ranging from 32 μM to 62.5 nM). Eighty five μl of binding assay buffer (20 mM HEPES, pH-7.5, 50 mM KCl, 5 mM MgCl₂, 20 mM sodium molybdate, 0.01% NP-40, 2 mM DTT, and 0.1 mg/ml BSA) was added to the wells of a 96 well black plate placed on ice. Three μl of 33.3 fold concentrated solutions of Hsp90 inhibitor compounds in DMSO, or DMSO alone (control) were added to the wells. Next, 10 μl either binding assay buffer alone (control) or human full length Hsp90 protein diluted in the binding assay buffer was added to achieve a final concentration of 50 nM. The plates were incubated at 4° C. on a plate shaker for 24 h. Two μl of 50× GM-FITC (9 nM final) in DMSO was added to all wells and the plate was incubated at ambient temperature for 1 h with shaking. Fluorescence polarization readings were taken in duplicate at excitation and emission wavelengths of 480 nm and 500 nm respectively. Specific binding was calculated by subtracting polarization values in the presence of Hsp90 from those in the absence of Hsp90. To calculate percent inhibition the specific binding values obtained for GA-FITC in the presence of compound were compared to values for GA-FITC in the absence of compound (DMSO only). Each plate had wells that contained a control compound, e.g. SNX-0723 at a concentration of 10 μM, 1 μM, or 80 nM, or the Hsp90 inhibitor at increasing concentrations.

Fluorescence polarization Hsp90 competitive binding assay characteristics herein resulted in IC₅₀ values for SBI-0638418 (Biogen-Idec, BIIB021) and SNX-0723 (Pfizer), that were consistent with values reported earlier, thus validating the assay. SBI-0638418 was determined to have an IC₅₀ of 20 nM, in agreement with the binding affinity reported in Mol. Canc. Ther. 8:921-929, 2009; and SNX-0723 was observed to have an IC₅₀ value of 30 nM, in agreement with the reported in J. Pharm. Exp. Ther. 332:849-857, 2010.

To assess assay precision, SNX-0723 was run as a control compound at one or two concentrations for each assay. SNX-0723 at 10 μM or 1 μM (n=12) consistently resulted inhibition values of 100%. At an SNX-0723 concentration of 84 nM (n=12) the inhibition values ranged from 77% to 100% (coefficient of variation, CV=9%). Further, a titration curve was obtained for SBI-0630353 in five assays (n=5), that resulted in IC₅₀ values ranging from 255 to 308 nM (CV=2%). These results showed that the run to run precision in the assay was high.

Precision of measurements within a run was assessed by CVs derived from duplicate determinations, and randomly compiled from five different assay plates (n=55). The range of CVs was 0-41%, with an average value of 11%.

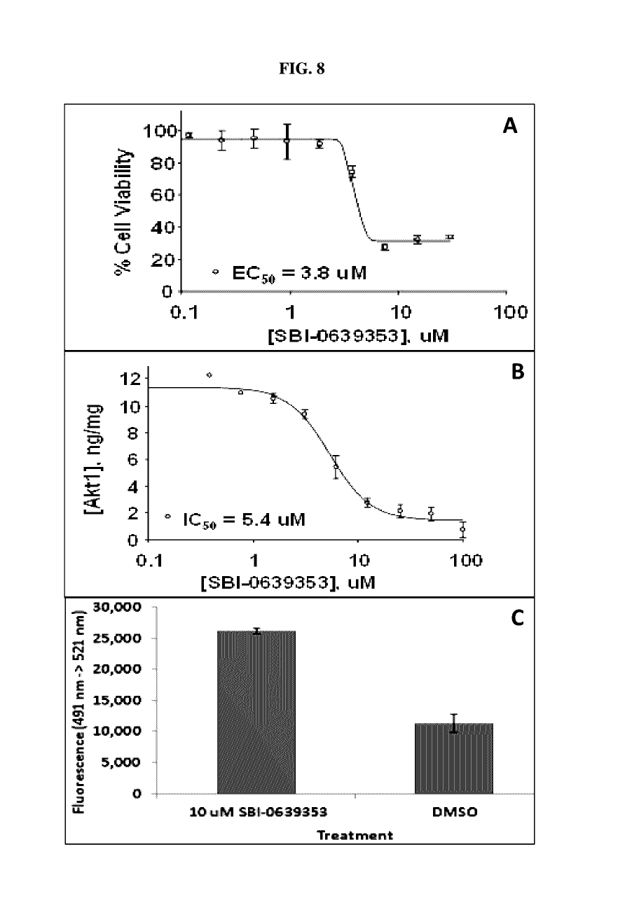

The IC₅₀ values for 58 Hsp90 inhibitors determined using the assay ranged from 97 nM to greater than 32 μM (Table II). The Kᵢ values ranged from 24 nM to greater than 8 μM. Representative concentration response binding curves are shown in FIG. 8. The binding affinities of a novel Hsp90 inhibitor demonstrated a clear structure activity relationship with the most potent inhibitors having Ki values under 100 nM.

TABLE II
Binding affinities of a novel Hsp90
inhibitor series ranked by potency.

| Compound | IC₅₀ (uM) | Kᵢ (uM) |
|---|---|---|
| SBI-0640613 | 0.097 | 0.024 |
| SBI-0640725 | 0.101 | 0.025 |
| SBI-0640610 | 0.194 | 0.049 |
| SBI-0639353 | 0.255 | 0.064 |
| SBI-0640608 | 0.311 | 0.078 |
| SBI-0640605 | 0.350 | 0.088 |
| SBI-0640607 | 0.368 | 0.092 |
| SBI-0640609 | 0.391 | 0.098 |
| SBI-0640606 | 0.432 | 0.108 |
| SBI-0639220 | 0.44 | 0.110 |
| SBI-0639354 | 0.72 | 0.180 |
| SBI-0639219 | 0.85 | 0.213 |
| SBI-0639350 | 1.10 | 0.275 |
| SBI-0640612 | 1.14 | 0.285 |
| SBI-0639217 | 1.20 | 0.300 |
| SBI-0639349 | 1.37 | 0.343 |
| SBI-0639218 | 1.47 | 0.368 |
| SBI-0640611 | 1.69 | 0.423 |
| SBI-0639899 | 2.15 | 0.538 |
| SBI-0639351 | 2.21 | 0.553 |
| SBI-0639355 | 2.87 | 0.718 |
| SBI-0206665 | 4.0 | 1.00 |
| SBI-0206664 | >10 | >2.5 |
| SBI-0630160 | >10 | >2.5 |
| SBI-0630161 | >10 | >2.5 |
| SBI-0630180 | >10 | >2.5 |
| SBI-0633823 | >10 | >2.5 |
| SBI-0633825 | >10 | >2.5 |
| SBI-0633826 | >10 | >2.5 |
| SBI-0634911 | >10 | >2.5 |
| SBI-0634912 | >10 | >2.5 |
| SBI-0635330 | >10 | >2.5 |
| SBI-0635446 | >10 | >2.5 |
| SBI-0635448 | >10 | >2.5 |
| SBI-0636373 | >10 | >2.5 |
| SBI-0636378 | >10 | >2.5 |
| SBI-0636436 | >10 | >2.5 |
| SBI-0636437 | >10 | >2.5 |
| SBI-0636438 | >10 | >2.5 |
| SBI-0636439 | >10 | >2.5 |
| SBI-0638965 | >10 | >2.5 |
| SBI-0638966 | >10 | >2.5 |
| SBI-0638967 | >10 | >2.5 |
| SBI-0638968 | >10 | >2.5 |
| SBI-0638969 | >10 | >2.5 |
| SBI-0638970 | >10 | >2.5 |
| SBI-0639179 | >10 | >2.5 |
| SBI-0639180 | >10 | >2.5 |
| SBI-0639181 | >10 | >2.5 |
| SBI-0639182 | >10 | >2.5 |
| SBI-0639186 | >10 | >2.5 |
| SBI-0640644 | 13.9 | 3.48 |
| SBI-0639901 | 30 | 7.50 |
| SBI-0640645 | 30 | 7.50 |
| SBI-0639900 | >32 | >8 |
| SBI-0639902 | >32 | >8 |
| SBI-0640492 | >32 | >8 |
| SBI-0639352 | >32 | >8 |

### Example 32

### Programmed Tumor Cell Death and Cellular Biomarker Assays

Tumor cell lines (LnCaP) were obtained from the Sanford Burnham Medical Research Institute (La Jolla, Calif.), LnCaP cells were cultured in culture medium (RPMI-glutamax, 10% FCS, 100 units/ml penicillin, and 100 μg/ml streptomycin). Cells were passaged after lifting using 0.25% trypsin/EDTA, and 3×10⁵ cells were seeded in each well of six well plates (total volume 2.5 mL/well). After the cells were cultured for 24 h, increasing concentrations of Hsp90 inhibitor were added to wells in triplicate from DMSO-containing stock solutions and mixed by gently by stirring. Final DMSO concentration in all wells was 0.25%. The treated cells were then cultured for 48 h prior to lysate preparation. The culture media was removed from wells, and the wells were washed twice with DPBS containing 1 mM CaCl₂ and 0.5 mM MgCl₂. Cells were then lysed in lysis buffer (PBS, 0.5% TX-100, 1 mM EDTA, 5 mM NaF, 1 mM sodium orthovanadate, 2.5 mM sodium pyrophosphate, and 1× HALT protease inhibitor.) One hundred μL of lysis buffer per well was used for 6 well plates and 40 μL was used for 12 well plates. Lysates were stored at −80° C. until assayed for Akt1. The protein concentration in the cell lysates was determined using the BCA protein assay kit used according to the manufacturer's recommendations. Twenty five μL of a 1:10 dilution of each lysate in PBS were added to wells of a 96 well plate. A standard curve was run by adding 25 μL of bovine serum albumin protein (provided with the BCA protein assay kit) dilutions ranging from 2.0-0.125 mg/ml. Two hundred μL of BCA Protein Assay Kit reagent was added and the mixtures were incubated at 37° C. for 30 min Protein concentrations were determined using a micro-titer plate reader (Model #Synergy HT; BioTek Inc., Winooski, Vt.).

Akt1 levels in the cell lysates were measured using kits from R&D Systems used according to the manufacturer's recommendations. Cell lysates were assayed at a 1:24 dilution for the Akt1 ELISA. The Akt1 concentrations in the cell lysates were extrapolated from the standard curve and corrected for lysate protein concentration. Akt1 levels were determined using a micro-titer plate reader (Model #Synergy HT; BioTek Inc., Winooski, Vt.). LnCaP cells were cultured in culture medium and passaged after lifting using 0.25% trypsin/EDTA. LnCaP cells were seeded into a 96 well tissue culture plate at 1.3×10⁴ cells per well in a volume of 100 uL of culture medium. After the cells were cultured for 24 h, increasing concentrations of Hsp90 inhibitor were added to wells in triplicate from DMSO containing stock solutions and mixed by gently by stirring. Final DMSO concentration in all wells was 0.25%. The cells were cultured for 48 h. Caspase 3/7 activity was measured using a Homogeneous Caspase 3/7 Assay Kit used according to the manufacturer's recommendations. In detail, an equal volume of Caspase 3/7 Assay Kit reagent was added to the wells and fluorescence readings (Ex/Em 485/528) were taken after 3, 6, and 23 h at ambient temperature using a micro-titer plate reader. A rhodamine 110 stock solution (10 mM) was prepared in DMSO and diluted with water within a range from 4000-62.5 nM. Fluorescence intensities of the dilutions were measured to obtain a standard curve. LnCaP cells were seeded into tissue culture plates using culture medium. After seeding, cultures were incubated for 16 h at 37° C. with 5% CO₂. Increasing concentrations of Hsp90 inhibitor were added to the culture from DMSO containing stock solutions and mixed gently by stirring. Final DMSO concentration in all wells was 0.25%. Cultures were plated in 386 well format and incubated for 72 h at 37° C. with 5% CO₂. Cell viability was measured with the ATPlite Kit used according to the manufacturer's recommendation. Cultures were equilibrated at ambient temperature for 30 min and 10 ul of ATPlite Kit reagent was added to each well. Cultures were mixed at 1,000 rpm for 2 min in the dark and luminescence quantification was accomplished using a micro-titer plate reader (POLARstar Omega micro-titer plate reader; BMG Labtech).

### Example 33

### Rat CNS Exposure, CNS Hsp90 Inhibition and CNS Biomarker Assay

Test subjects (Sprague-Dawley rats) were housed in sterile vivarium with controlled temperature, humidity and 12-hour light-dark cycle (7:00 lights-on and 19:00 lights-off). Following procurement, rats were acclimated to the vivarium facility for 7 days prior to study day-1 with ad libitum access to both diet and water. Bedding was changed twice weekly. Test articles were formulated in 100% PEG400 at concentrations of 16 mg/mL on study day-1 for the 40 mg/Kg doses. On study day-1 rats were administered vehicle or Hsp90 inhibitor formulation. Following administrations of test article formulation, blood and CNS tissue was collected at 6.5 h post-dosed by a necropsy procedure. In detail, CNS tissue was isolated from the Vehicle and dose groups, divided into 2 mid-sagittal sections, placed in tarred 1 mL micro-centrifuge tubes, weighed and immediately frozen on dry ice. Blood was isolated by a cardiac puncture procedure from the Vehicle and Hsp90 inhibitor dose groups. From these samples, plasma was isolated in plasma separator tubes containing ethylenediaminetetraacetic acid as the anticoagulant. HSP90 inhibitor concentrations in plasma and CNS tissue were determined using LC-MS/MS based methods following a 60% acetonitrile extraction. Hsp90 binding sites in CNS lysates in fluorescently were quantified by labeled geldanamycin displacement assays (see Example 31). Quantification of Akt1 in CNS lysates was accomplished by ELISA based assays (see Example 32).

### Example 34

### Hsp70 Induction in the CNS as a Biomarker for Therapeutic Benefits of Hsp90 Inhibition

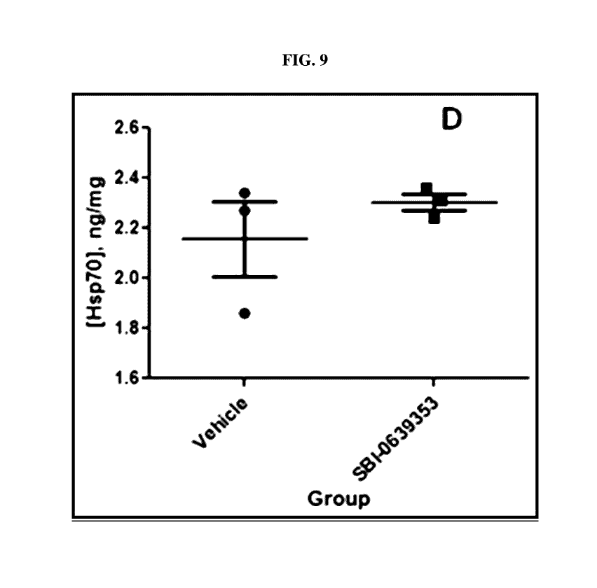

One of the effects of Hsp90 inhibition is an increase in the levels of another molecular chaperone known as Heat Shock Protein-70 (Hsp70), which is also a biomarker. FIG. 9 shows that Hsp70 level increases in rats treated with the control compound SNX-0723 compared to untreated rats (vehicle) and remains elevated up to 24 hrs (X-axis) post dosing. Increased level of Hsp70 upon inhibition of Hsp90 has a therapeutic benefit for the treatment of neurodegenarative disorders, including PD, AD, Amyotrophic Lateral Sclerosis (ALS), Huntington's disease and multiple sclerosis.

Quantification of total CNS tissue Hsp70 was accomplished by a sandwich ELISA assay kit (catalog #DYC1663, R & D systems Inc.) used according to the manufacturer's recommendations. In general the procedure for quantification of Hsp70 in CNS is as follows. Brain tissue samples from experimental animals treated with compounds of formula (I) herein or with vehicle alone are homogenized in PBS buffer and centrifuged at 15,000 g for 30 minutes and the supernatant collected and stored at −80° C. Before use, the samples are removed and placed on ice for thawing, following which they are centrifuged at 2000×g for 5 minutes, and the supernatant transferred to a clean test tube. Sample protein concentrations are quantified using a total protein assay.

Hsp70 specific capture antibody is diluted to the working concentration as recommended in the product. A 96-well microplate is coated with 100 μL per well of the diluted capture antibody. The plate is sealed and incubated overnight at room temperature. Each well is aspirated and washed with Wash Buffer, and the process repeated two times for a total of 3 washes. Each was uses 400 μL of Wash Buffer. Wells of the plate are blocked by adding 300 μL of Block Buffer to each well and incubating at room temperature for 1-2 hours. The aspiration/wash step is repeated as in step 2. The plates are now ready for sample addition. Sample or standards (100 μL) diluted in an appropriate diluent is added to the wells. The plate is covered with an adhesive strip and incubated for 2 hours at room temperature. The aspiration/wash step is repeated. Next 100 μL of a detection antibody, diluted in a suitable diluent is added to each well, and the plate covered with a new adhesive strip and incubated for 2 hours at room temperature. The wells are next washed as in previous steps. Streptavidin-HRP is diluted to the recommended working concentration, and 100 μL of the diluted Streptavidin-HRP is added to each well. After an incubation period of 20 minutes at room temperature, the aspiration/wash step, as in step 2, is repeated. Next 100 μL of a solution of HRP substrate is added to each well, followed by 20 minutes incubation at room temperature. In the following step 50 μL of Stop Solution is added to each well, followed by gentle mixing. Optical density of each well is immediately measured, using a microplate reader set to 450 nm. Increased level of Hsp70 upon inhibition of Hsp90 has a therapeutic benefit for the treatment of neurodegenarative disorders, including PD, AD, Amyotrophic Lateral Sclerosis (ALS), Huntington's disease and multiple sclerosis.

### Example 35

### Determination of the Antifungal Activity

The antifungal activity of the compounds of the formula (1) is determined as follows. The compounds are tested against a panel of fungi including Candida parapsilosis, Candida tropicalis, Candida albicans-ATCC 36082 and Cryptococcus neoformans. The test organisms are maintained on Sabourand Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The antifungal activity of a test compounds is determined using a modification of a broth microdilution technique. Test compounds are diluted in DMSO to a 1.0 mg/ml ratio, then diluted to 64 ug/ml in YNB broth, pH 7.0 with MOPS (Fluconazole is used as the control) to provide a working solution of each compound. Using a 96-well plate, wells 1, and 3 through 12 are prepared with YNB broth. Ten-fold dilutions of the test compound solution are made in wells 2 to 11 (concentration ranges are 64 to 0.125 μg/ml). Well 1 serves as a sterility control, and blank for the spectrophotometric assays. Well 12 serves as a growth control. The microtitre plates are inoculated with 10 μl of the blastospore suspension in each of wells 2 to 11 (final inoculum size is 10⁴ organisms/ml). Inoculated plates are incubated for 48 hours at 35° C. The minimum inhibitory concentration (MIC) values are determined spectrophotometrically by measuring the absorbance at 420 nm (Biotek Synergy plate reader.) after agitation of the plates for 2 minutes with a vortex-mixer (Vorte-Genie 2 Mixer, Scientific Industries, Inc., Bolemia, N.Y.). The MIC endpoint is defined as the lowest drug concentration exhibiting approximately 50% (or more) reduction of the growth compared with the control well. With the turbidity assay this is defined as the lowest drug concentration at which turbidity in the well is <50% of the control. Minimal Cytolytic Concentrations (MCC) are determined by sub-culturing all wells from the 96-well plate onto a Sabourand Dextrose Agar (SDA) plate, incubating for 1 to 2 days at 35° C., and then checking viability. This approach can be used to test the potency of compounds for treating antifungal infections caused by a broad class of fungus.

### Example 36

### Methods of Testing for Pain Reducing or Pain Preventing Activity

(I) Inflammatory Hyperalgesia Test: Mechanical hyperalgesia can be examined in a rat model of inflammatory pain. Thresholds of paw withdrawal to an increasing pressure stimulus are measured by the Randal-Sellito technique using an analgesymeter (Ugo Basile, Milan), in naïve animals prior to an intraplantar injection of complete Freund's complete adjuvant (FCA) into the left hind paw. Paw withdrawal thresholds are measured again 24 hours later prior to (predose) and then from 10 minutes to 6 hours following the administration of compounds of formula (I) herein or vehicle alone. Reversal of hyperalgesia in the ipsilateral paw is calculated according to the formula:

$$\% reversal = \frac{post dose threshold - predose threshold}{naive threshold - predose threshold} \times 100$$

(ii) Neuropathic hyperalgesia test: Mechanical hyperalgesia can be examined in a rat model of neuropathic pain induced by partial ligation of the left sciatic nerve. Approximately 14 days following surgery mechanical withdrawal thresholds of both the ligated (ipsilateral) and non-ligated (contralateral) paw are measured prior to (predose), and then from 10 minutes to 6 hours following administration of compounds of formula (I) herein or vehicle alone. Reversal of hyperalgesia at each time point is calculated according to the formula:

$$\% reversal = \frac{\begin{matrix}ipsilateral threshold postdose - \\ ipsilateral threshold predose\end{matrix}}{\begin{matrix}contralateral threshold predose - \\ ipsilateral threshold predose\end{matrix}} \times 100$$

Tests above are carried out using groups of six animals. Stock concentrations of drugs are dissolved in distilled water, and subsequent dilutions are made in 0.9% saline for subcutaneous administration in a volume of 4 ml/kg. Compounds herein are dissolved in plastic vials, and kept in the dark.

Statistical analysis are carried out on withdrawal threshold readings (g) using ANOVA with repeated measures followed by Tukey's HSD test. Efficacy refers to the maximal reversal of hyperalgesia observed at the doses used.

(iii) Testing the effect of compounds of formula (1) in a Rat Model of Bone Cancer Pain: Adult female rats are given intra-tibial injections of MRMZ-1 rat mammary gland carcinoma cells (3 μl, 10⁷ cells/ml). Typically the animals typically gradually develop mechanical hyperalgesia, mechanical allodynia (skin sensitivity to non-noxious stimuli) and hind limb sparing, beginning on day 12-14 following cell injection. A compound of formula (1) (e.g. at a dose of 10 and 30 ug/kg s.c.) is administered 3 times a week from the day of cell injection, and the extent of inhibition of hind limb sparing and mechanical allodynia is determined in comparison to vehicle-treated controls.

The approach above can be used to treat pain related disorders and inflammations of various types.

### Example 37

### Parasite in Vitro Differentiation and Manipulation

The ability of the compounds of formula (1) herein to inhibit in vitro differentiation of a parasite is determined using the following method.

RH uracil phosphoribosyltransferase (UPRT) knock-out parasites can be induced to differentiate into bradyzoites in low CO₂, resulting in pyrimidine starvation. (Bohne et al., (eds) (1997) Stage-specific expression of a selectable marker in Toxoplasma gondii permits selective inhibition of either tachyzoites or bradyzoites Vol. 88. Mol Biochem Parasitol; Bohne et al., (1997) Mol Biochem Parasitol 88, 115-126). CO₂ depletion is accomplished by inoculating tachyzoites with low inocula (parasite/host cell ratio<1:10) into a human foreskin fibroblast (HFF) host cell monolayer in minimal essential medium (Dulbecco's modified Eagle's medium, DMEM) with 10% FBS (Gibco® Cell Culture Products, Invitrogen, Carlsbad, Calif.) without NaHCO₃ but containing 25 mm HEPES. Cultures of parasites are equilibrated at pH 7 and incubated at 37° C. at ambient CO₂ (0.03%). In other experiments, compounds of the formula (1) (100 nM) or DMSO (as a control) are added to the same media and conditions. By about 4 days, the vacuoles show distinct signs of becoming cysts: parasite division is reduced and cyst wall is evident (Bohne et al., (eds) (1997) Stage-specific expression of a selectable marker in Toxoplasma gondii permits selective inhibition of either tachyzoites or bradyzoites Vol. 88. Mol Biochem Parasitol; Bohne et al., (1997) Mol Biochem Parasitol 88, 115-126). Bradyzoite induction under this method is assessed and followed by cyst wall detection using the Dolichos biflorus lectin (Boothroyd et al., (1997) Philos Trans R Soc Loud B Biol Sci 352, 1347-1354).

To induce PK tachyzoites, a clone isolated from cystogenic T. gondii Me49 strain (Kasper et al., (1985) J Clin Invest 75, 1570-1577), to differentiate to bradyzoites in vitro, the high-pH method is chosen (Soete et al., (1994) Exp Parasitol 78, 361-370). A confluent monolayer of HFF is infected with approximately 2×10⁵ tachyzoites in each well of a 24-well plate or 10×10⁶ in 8 cm diameter tissue culture petri dish and are grown in standard tachyzoite conditions for 4 h at pH 7.2, under 5% CO2 to permit invasion and initial growth. After this, the medium is removed and replaced with inducing medium (RPMI/HEPES, pH 8.1, 5% fetal bovine serum) and the culture placed in a 37° C. incubator (at ambient CO2 0.03%). In other experiments, compounds of the formula (I) herein (100 nM) or DMSO (as a control) are added to the same media and conditions. The inducing medium is replaced every 2nd day. By about 2 days, the vacuoles show distinct signs of becoming cysts (rounding up and showing packed parasites, compared with the flattened rosettes of the tachyzoite vacuoles) and parasite division rate is reduced. Antibodies specific to the tachyzoite surface protein SAG1 (murine mAb α-p30 T4IE5) or to the bradyzoite specific protein P34 (murine mAb α-34 T82C2) or P21 (murine mAb T84G10) (Tomavo et al., 1991 Infect Immun 59, 3750-3753), as well as D. biflorus lectin (Sigma, St Louis, Mo.), are used to control bradyzoite development.

In both models for bradyzoite isolation, bradyzoite induction medium is removed, cells are washed once with PBS, the monolayer is scraped and passed five times through a 27-gauge needle, followed by once through a 30-gauge needle to release parasites from the host cells. The parasites are then centrifuged at 1800 r.p.m. for 10 min at room temperature and resuspended in sterile PBS and counted in a Neubauer improved chamber. Tachyzoite cultures can be obtained from growing parasites in standard tachyzoite conditions and processed similarly except that for release from the HFFs a 27-gauge needle is used. Both stages of parasites are purified from the host cell material by passage through a 3 um-pore size filter (Nucleopore Corporation, Pleasanton, Calif.).

This approach can be used to treat infections caused by parasites that cause malaria and systemic toxoplasmosis.

### Example 38

### Effect of SBI-0640725 on Cell Viability, Client Protein Stability, Hsp 70 Induction Mitochondrial Stress Response, and Mitochondrial Integrity

In this Example, the effect of SBI-0640725 on a number of cellular parameters was measured in U251MG (human glioblastoma astrocytoma), MBA-MD-231 (triple negative breast cancer), and HepG2 (liver) cells.

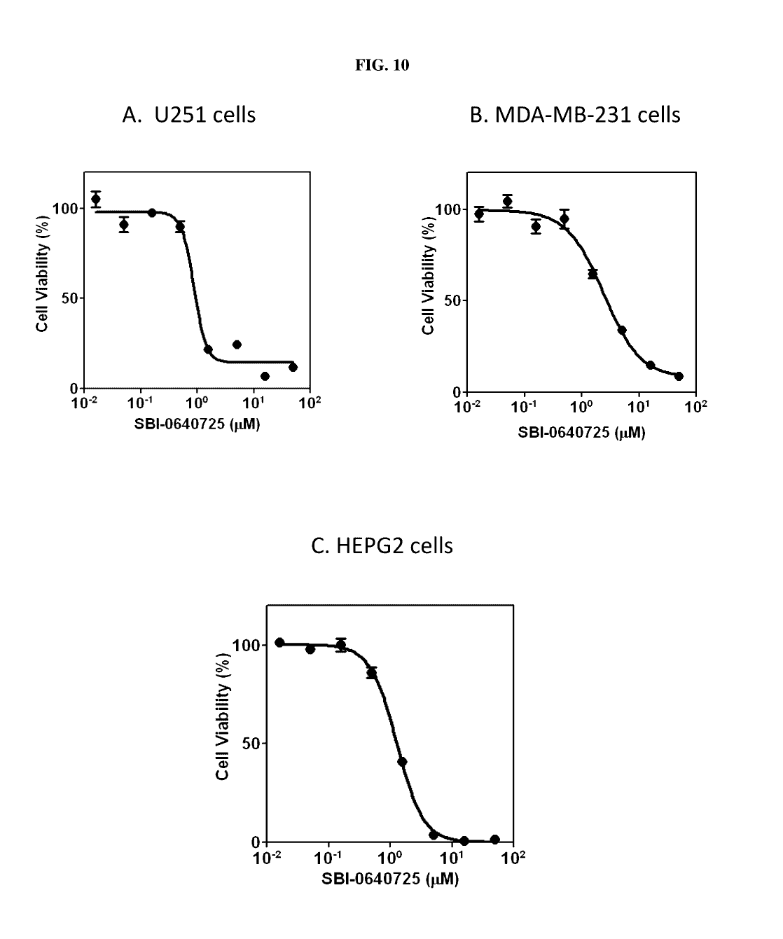

The effect of SBI-0640725 on cell viability was determined by measuring cellular dehydrogenase activity (U251MG and MBA-MD-231 cells) or by measuring ADP levels (HepG2) after treating cells for 72 hours with increasing concentrations of SBI-0640725. To measure dehydrogenase activity, CCK-8 kit reagent (Dojindo) was added to the wells and incubated for 2 hours, after which absorbance was measured at 450 nm using a Synergy HT micro-titer plate reader (Biotek). To measure ADP levels, ADP-GLO reagent (Life Technologies) was added to the wells, and luminescence was measured after a 30 minute incubation using a POLARstar Omega micro-titer plate reader (BMG). As shown in FIG. 10 panels A, B and C, treatment with SBI-0640725 results in a reduction in cell viability for U251MG, MBA-MD-231 and HepG2 cells, respectively.

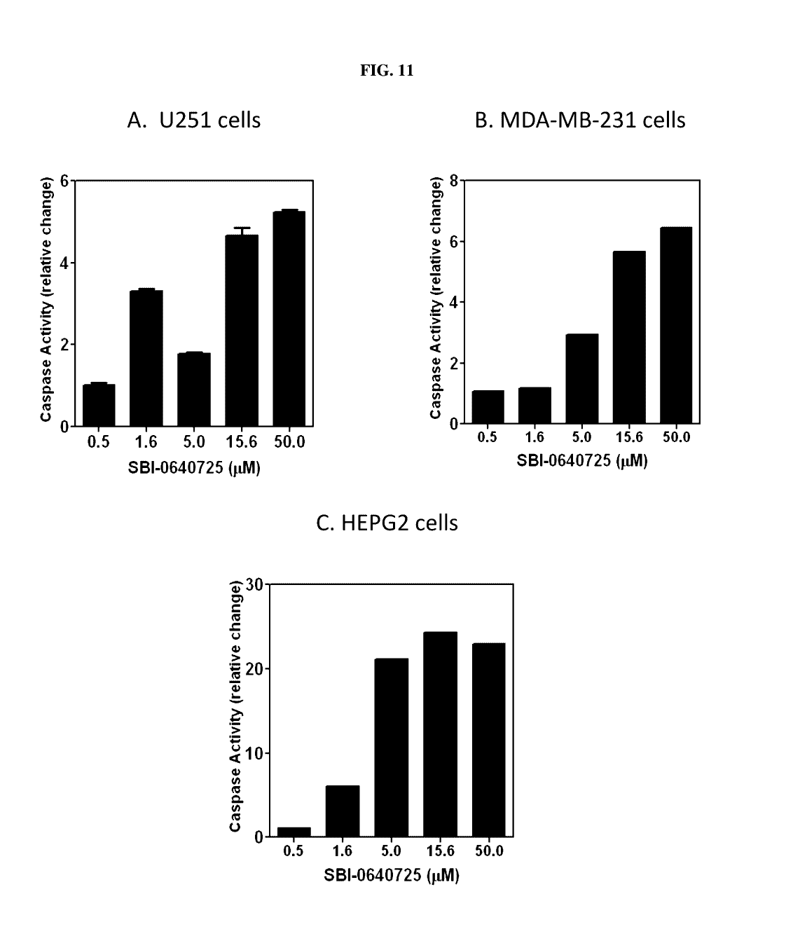

Further, the effect of SBI-0640725 on cellular caspase levels was also quantitated in U251MG, MBA-MD-231 and HepG2 cells. Each cell type was treated with increasing concentrations of SBI-0640725 for 72 hours. Quantification of total cellular Caspase-3 and 7 activity was accomplished using a Caspase-3/7 luminescence assay kit (Promega) according to the manufacturer's recommendations, and analyzed with a POLARstar Omega micro-titer plate reader (BMG). As shown in FIG. 11 panels A, B and C, treatment with SBI-0640725 resulted in an increase in Caspase 3/7 activity in U251MG, MBA-MD-231, and HepG2 cells, respectively. Caspase activity is a marker for apoptotic cell death. Increase in Caspase activity in response to treatment with SBI-0640725 correlates well with the cell viability data (FIG. 10), thus indicating that SBI-0640725 is associated with an apoptotic mechanism of cell death.

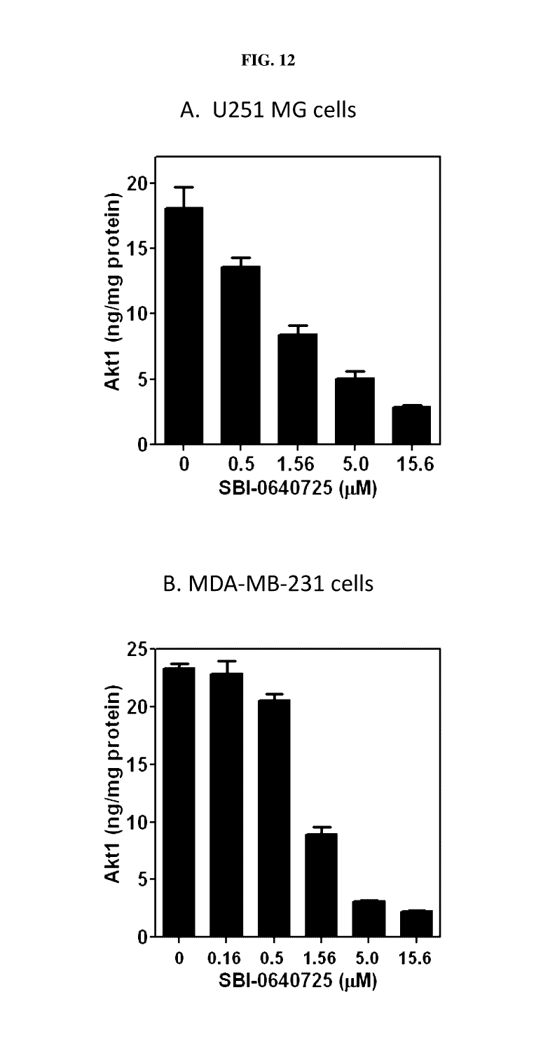

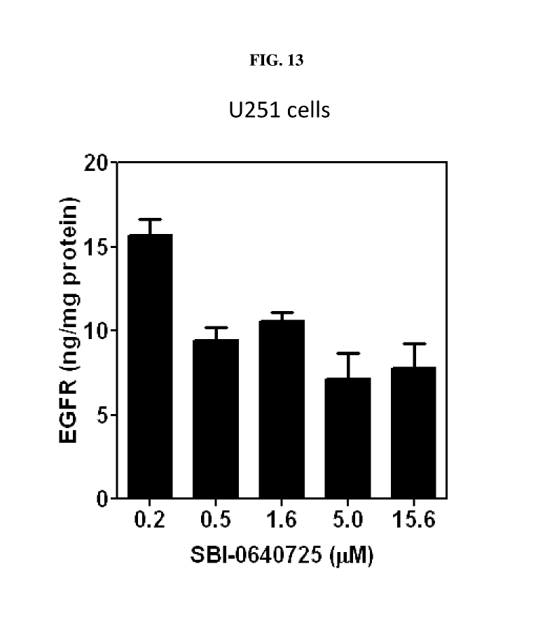

In further experiments, the effect of SBI-0640725 on the stability of Hsp90 client proteins was analyzed. It was observed that in both U251MG (FIG. 12 panel A) and MBA-MD-231 cells (FIG. 12 panel B), after treatment for 48 hours with increasing concentrations of SBI-0640725, there was a progressive decrease in the levels of the client protein Akt1. Akt1 protein levels were measured in cell lysates using ELISA. Additionally, the level of the client protein EGFR was observed to decrease in U251MG cells treated for 48 hours with increasing concentrations of SBI-0640725 (FIG. 13).

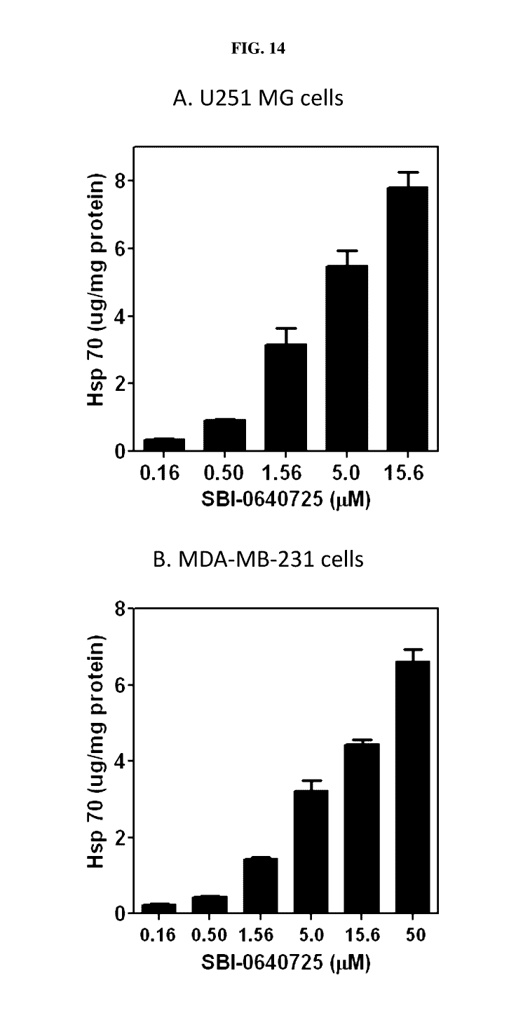

Experiments were performed using U251 MG and MBA-MD-231 cells to also measure induction of Hsp 70 in response to treatment with SBI-0640725. Hsp70 has been found to be beneficial for neurological indications, in which it appears to confer a protective effect. It was observed that the compound caused induction of Hsp70 in each cell type (see FIG. 14, A and B). For Hsp70 induction measurements, cells were treated with increasing concentrations of SBI-0640725 for 48 hours, and Hsp70 levels were measured in cell lysates using ELISA.

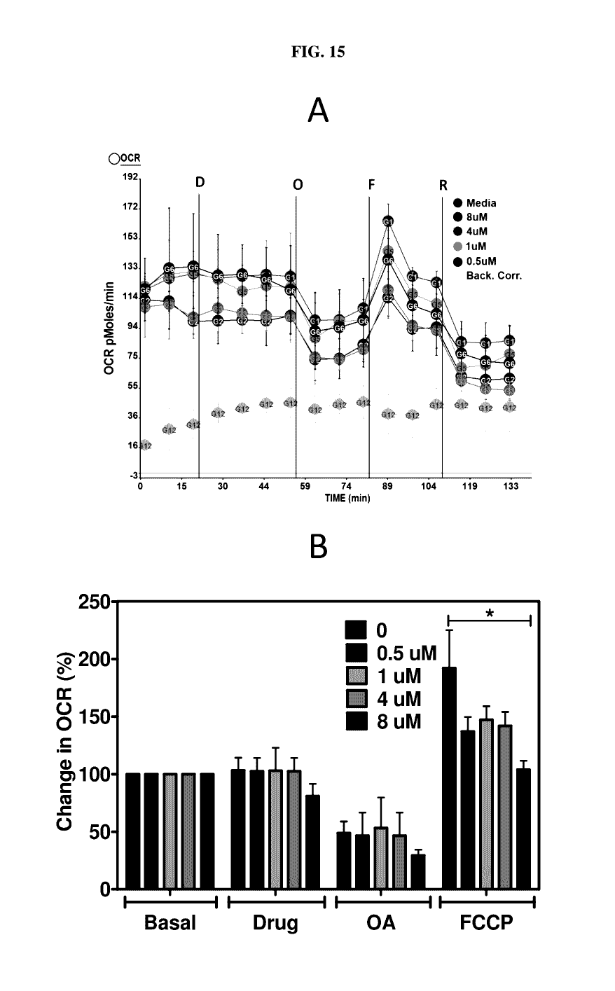

In further experiments, the effect of SBI-0640725 on mitochondrial stress response in U251MG cells was determined by measuring the mitochondrial oxygen consumption rate (OCR). The OCR was measured in real time, both before, and after treating U251 MG cells with increasing concentrations of SBI-0640725. The time of compound addition is indicated in FIG. 15A by the vertical line labeled D. Mitochondrial bioenergetics stress was determined by treating the cells sequentially with 0.125 mg/ml oligomycin (OA; vertical line O), 0.5 mM carbonyl cyanide-4-(trifluoromethoxy)phenylhydrazone (FCCP; vertical line F) and 1 mM rotenone (vertical line R) while continuing OCR measurement. Oligomycin (OA) generates mitochondrial stress by inhibiting ATP synthase and FCCP uncouples mitochondrial oxidative phosphorylation. The OCR data shown in FIG. 15A is presented in FIG. 15B after normalization using cell protein concentration, and subtracting extra-mitochondrial respiration, as determined by the respiration observed upon addition of rotenone. These experiments tested whether SBI-0640725 could inhibit the ability of mitochondria to compensate for these stressors. SBI-0640725 was observed to significantly inhibit the ability of mitochondria to compensate for stress when FCCP was used as an inducer of stress. FCCP dissipates the electrochemical gradient that drives ATP synthesis. Thus, in order to maintain the membrane potential, mitochondria needs to increase the flow of electrons, thereby reflecting an increase in the OCR. SBI-0640725 significantly inhibited this response to stress by the mitochondria.

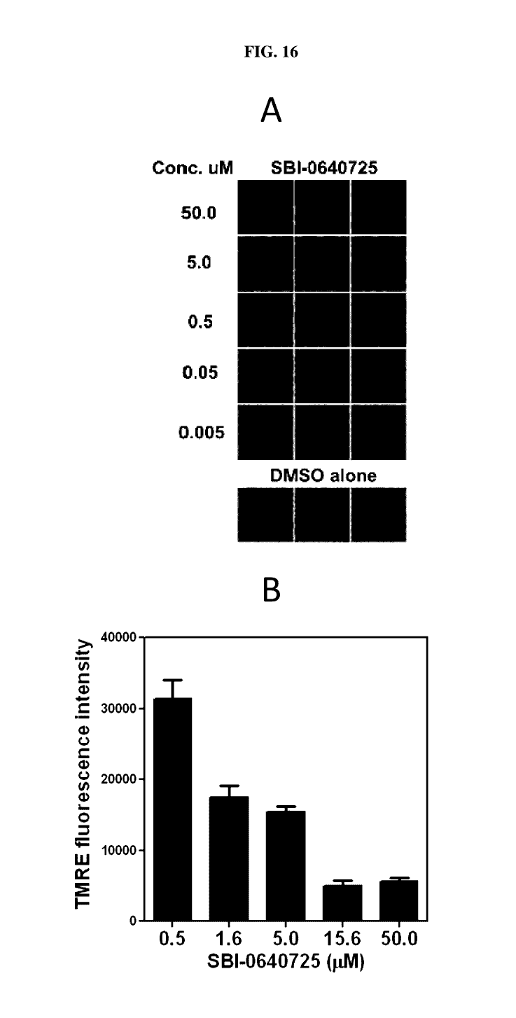

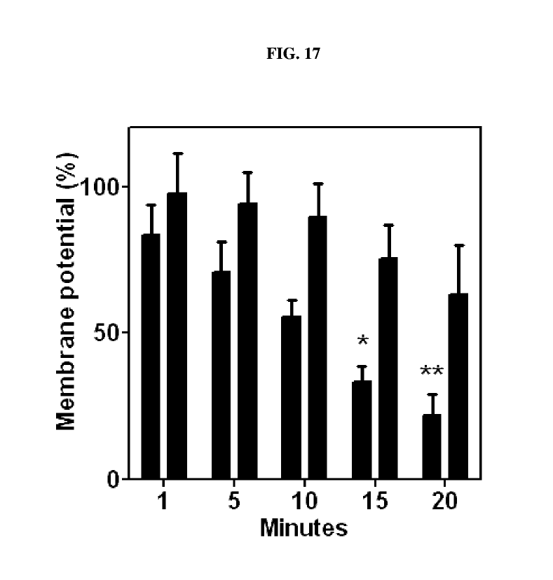

Additional experiments were performed to determine the effect of SBI-0640725 on mitochondrial integrity. U251MG cells were treated with tetramethylrhodamine ethyl ester (TMRE), and fluorescence was visualized using fluorescence confocal microscopy (FIG. 16 panel A), or quantitated using a fluorimeter (FIG. 16 panel B) after cells had been treated with increasing concentrations of SBI-0640725 for 48 hours. Mitochondrial integrity, as a measured by the ability of mitochondria to retain de-esterified TMRE, was observed to decrease proportionately with increasing concentrations of SBI-0640725. In experiments using isolated mitochondria, mitochondrial membrane potential was determined as a measure of mitochondrial integrity. TMRE-loaded mitochondria isolated from U251MG cells were treated with either 10 μM (left bar of the pair in FIG. 17) or 2 μM (right bar of the pair in FIG. 17) SBI-0640725 while continually monitoring changes in fluorescence intensity at 30° C. It was observed that with time the mitochondrial membrane potential decreased in SBI-0640725 treated cells, and the decrease was greater in cells treated with the higher concentration of SBI-0640725. These results also show that SBI-0640725 causes a decrease in mitochondrial integrity.

The table below summarizes efficacy of SBI-0640725 in various cancer cell lines, including U251MG, MDA-MB-231, and HepG2, using ADP-GLO assay described above. As used herein “efficacy” (EC₅₀) is the concentration of a compound that produces half-maximal response in a cellular assay.

| | | | | Liver | Ovarian | |
|---|---|---|---|---|---|---|
| Indication | Breast Cancer | GBM | | Cancer | Cancer | |
| Cell Line | MDA-MB-231 | U251MG | U87MG | HepG2 | R127 | R182 |
| EC₅₀ (uM) | 2.4 | 0.88 | 6.7 | 1.3 | 0.41 | 0.69 |

1. A compound of the formula 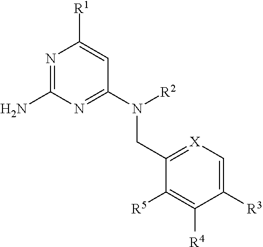
or a salt, hydrate, or solvate thereof, wherein
R¹ is H; CH₃; OCH₃; CF₃; F; Cl; Br; or I;
X is C; or N;
R² is H; CH₃; CH2CH₃; CH(CH₃)₂; C(CH₃)₃; CH₂CH₂CH₃; CH₂CH₂CH₂OH; CH₂CH₂CH₂CH₃; CH₂CH₂CH₂CH₃; CH₂CHCH₂; CH₂CH₂CH₂CH₂CH₂CH₃; 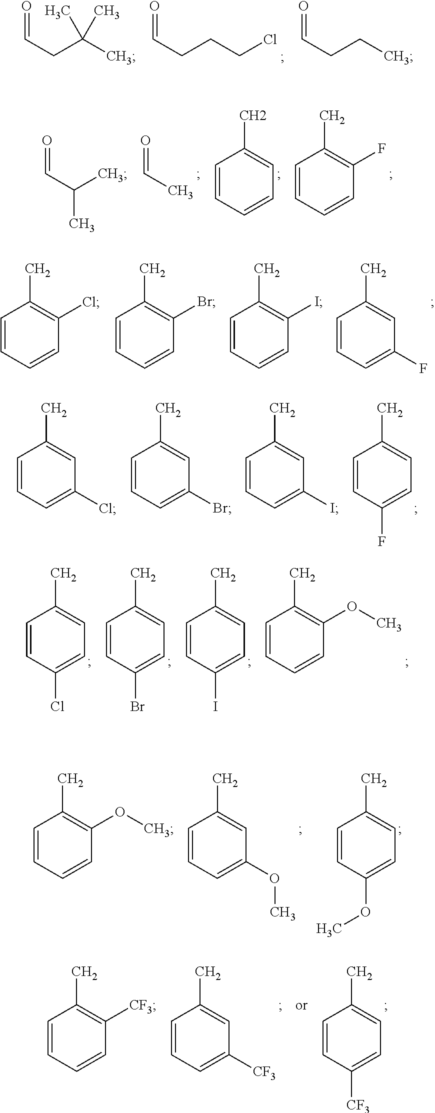
R³ is H; CH₃; OCH₃; F; Cl; Br; I; or CF₃;
R⁴ is H; CH₃; OCH₃; F; Cl; Br; I; or CF₃;
R⁵ is H; CH₃; OCH₃; F; Cl; Br; I; or CF₃.

2. The compound according to claim 1 wherein R² is

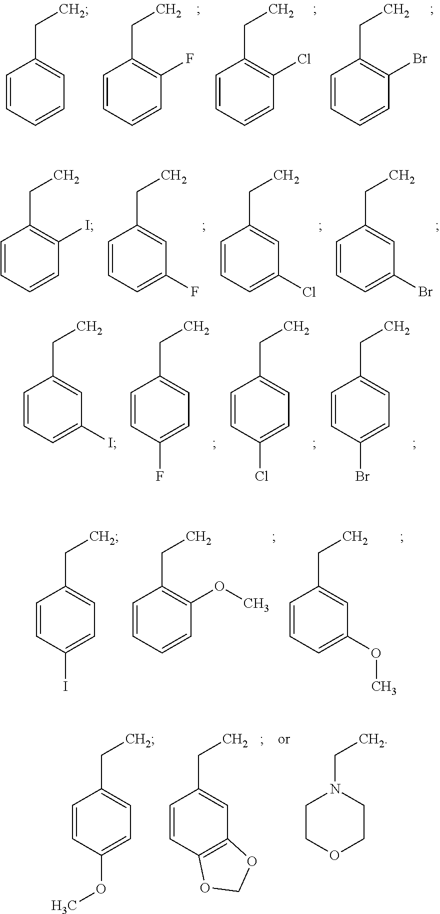

3. A pharmaceutical composition comprising at least one compound according to claim 1, together with one or more pharmaceutically acceptable carriers or excipients.

4. A method for prophylaxis or treatment of a disease state or condition in a subject, wherein the disease state or condition is responsive to inhibition of Hsp90 activity in the subject, the method comprising administering to the subject in need thereof, an amount of at least one compound according to claim 1 effective to inhibit the Hsp90 activity.

5. (canceled)

6. A method for prophylaxis or treatment of a disease state or condition in a subject, wherein the disease state or condition is responsive to inhibition of Hsp90 activity in the subject, the method comprising administering to the subject in need thereof, an amount of at least one compound according to claim 1 effective to inhibit the Hsp90 activity, and an additional therapeutic agent.

7-11. (canceled)

12. A method for prophylaxis or treatment of a disease state or condition in a subject undergoing treatment with a therapeutic agent, wherein the disease state or condition is the development of resistance to the therapeutic agent, wherein the disease state or condition is responsive to inhibition of Hsp90 activity in the subject, the method comprising: administering to the subject in need thereof, an amount of at least one compound according to claim 1 effective to inhibit the Hsp90 activity.

13-15. (canceled)

16. The method according to claim 4, wherein the Hsp90-mediated disease state or condition is selected from the group comprising an autoimmune disease, an inflammatory disease, a neurological disease, an infection, a cancer, a carcinoma, a cardiovascular disease, an allergy, asthma, a proliferative disorder, a metabolic disease, a leukemia, a neoplasm, a hormone-related disease, age-related macular degeneration, and, tumors or symptoms resulting from neurofibromatosis.

17. (canceled)

18. The method according to claim 4, wherein the Hsp90-mediated disease or condition is a neurodegenerative disease selected from the group comprising Parkinson's disease, Alzheimer's disease, Huntington's disease, and Amyotrophic lateral sclerosis.

19-25. (canceled)

26. A method for prophylaxis or treatment of a disease or condition comprising or arising from abnormal cell growth in a mammal, the method comprising administering to the mammal an amount of at least one compound according to claim 1 effective to inhibit Hsp90 activity in the mammal.

27. (canceled)

28. The method according to claim 26, wherein the disease or condition comprising or arising from abnormal cell growth comprises a carcinoma of the bladder, breast, colon, kidney, epidermis, liver, lung, esophagus, gall bladder, ovary, pancreas, stomach, cervix, thyroid, prostate, gastrointestinal system, or skin; a hematopoietic tumor of lymphoid lineage; a hematopoietic tumor of myeloid lineage; thyroid follicular cancer; a tumor of mesenchymal origin; a tumour of the central or peripheral nervous system; melanoma; seminoma; teratocarcinoma; osteosarcoma; xeroderma pigmentosum; neurofibromatosis; keratoacanthoma; thyroid follicular cancer; and Kaposi's sarcoma.

29. A method for alleviating or reducing the incidence of resistance to an anticancer drug comprising administering to a subject in need thereof, an amount of at least one compound according to claim 1 effective to inhibit the Hsp90 activity in the subject.

30-36. (canceled)

37. A pharmaceutical composition comprising at least one compound according to claim 2, together with one or more pharmaceutically acceptable carriers or excipients.

38. A method for prophylaxis or treatment of a disease state or condition in a subject, wherein the disease state or condition is responsive to inhibition of Hsp90 activity in the subject, the method comprising administering to the subject in need thereof, an amount of at least one compound according to claim 2 effective to inhibit the Hsp90 activity.

39. A method for prophylaxis or treatment of a disease state or condition in a subject, wherein the disease state or condition is responsive to inhibition of Hsp90 activity in the subject, the method comprising administering to the subject in need thereof, an amount of at least one compound according to claim 2 effective to inhibit the Hsp90 activity, and an additional therapeutic agent.

40. A method for prophylaxis or treatment of a disease state or condition in a subject undergoing treatment with a therapeutic agent, wherein the disease state or condition is the development of resistance to the therapeutic agent, wherein the disease state or condition is responsive to inhibition of Hsp90 activity in the subject, the method comprising: administering to the subject in need thereof, an amount of at least one compound according to claim 2 effective to inhibit the Hsp90 activity.

41. The method according to claim 38, wherein the Hsp90-mediated disease state or condition is selected from the group comprising an autoimmune disease, an inflammatory disease, a neurological disease, an infection, a cancer, a carcinoma, a cardiovascular disease, an allergy, asthma, a proliferative disorder, a metabolic disease, a leukemia, a neoplasm, a hormone-related disease, age-related macular degeneration, and, tumors or symptoms resulting from neurofibromatosis.

42. The method according to claim 38, wherein the Hsp90-mediated disease or condition is a neurodegenerative disease selected from the group comprising Parkinson's disease, Alzheimer's disease, Huntington's disease, and Amyotrophic lateral sclerosis.

43. A method for prophylaxis or treatment of a disease or condition comprising, or arising from abnormal cell growth in a mammal, the method comprising administering to the mammal an amount of at least one compound according to claim 2, effective to inhibit Hsp90 activity in the mammal.

44. The method according to claim 43, wherein the disease or condition comprising or arising from abnormal cell growth comprises a carcinoma of the bladder, breast, colon, kidney, epidermis, liver, lung, esophagus, gall bladder, ovary, pancreas, stomach, cervix, thyroid, prostate, gastrointestinal system, or skin; a hematopoietic tumor of lymphoid lineage; a hematopoietic tumor of myeloid lineage; thyroid follicular cancer; a tumor of mesenchymal origin; a tumour of the central or peripheral nervous system; melanoma; seminoma; teratocarcinoma; osteosarcoma; xeroderma pigmentosum; neurofibromatosis; keratoacanthoma; thyroid follicular cancer; and Kaposi's sarcoma.

45. A method for alleviating or reducing the incidence of resistance to an anticancer drug comprising administering to a subject in need thereof, an amount of at least one compound according to claim 2 effective to inhibit the Hsp90 activity in the subject.
